(19) United States
(12) Patent Application Publication
(10) Pub. No.: US 2013/0172741 A1
(43) Pub. Date: Jul. 4, 2013

(54) ANALYSIS APPARATUS AND ANALYSIS METHOD

(71) Applicants: Koichi Kusukame, Nara (JP); Tatsuo Itoh, Osaka (JP); Hiromichi Horinaka, Osaka (JP)

(72) Inventors: Koichi Kusukame, Nara (JP); Tatsuo Itoh, Osaka (JP); Hiromichi Horinaka, Osaka (JP)

(21) Appl. No.: 13/820,788

(22) PCT Filed: Jul. 9, 2012
(86) PCT No.: PCT/JP2012/004426

§ 371 Date: Mar. 5, 2013

(30) Foreign Application Priority Data
Jul. 14, 2011 (JP) 2011-156120
Jul. 14, 2011 (JP) 2011-156121

(51) Int. Cl. A61B 5/01; A61B 5/00; A61B 8/08

(52) U.S. Cl. CPC A61B 5/01; A61B 8/48; A61B 5/0075
USPC 600/438; 600/549; 600/476

(57) ABSTRACT

An analysis apparatus which analyses a state of a specimen and includes: a temperature adjustment unit which lowers a temperature of the specimen by cooling the specimen; a light source which heats at least a part of the specimen cooled by the temperature adjustment unit, by illuminating the specimen with light; a first temperature measurement unit which measures a temperature change of the specimen caused by the heating by the light source; and an analysis unit which analyzes the state of the specimen based on the temperature change of the specimen. For example, the first temperature measurement unit has an ultrasonic probe which transmits an ultrasonic pulse to the specimen and receives a reflected wave that is the ultrasonic pulse reflected from the specimen, and measures the temperature of the specimen based on a signal of the reflected wave.

### TECHNICAL FIELD

The present invention relates to an analysis apparatus which analyzes a state of a specimen, and an analysis method for analyzing a state of a specimen.

### BACKGROUND ART

Spectroscopic measurement apparatuses which measure optical absorption characteristics inside a body tissue are capable of measuring concentration distributions of various constituents utilizing optical absorption characteristics (a relationship between a wavelength of light and an optical absorption) that vary from material to material, and are used for medical diagnosis in various fields. For example, the spectroscopic measurement apparatuses are capable of measuring concentration distributions of oxygenated hemoglobin and deoxygenated hemoglobin inside the body and determining on a formation of a new blood vessel and on an oxygen saturation of hemoglobin which are associated with tumor growth, and are utilized for diagnosis. Such spectroscopic measurement apparatuses are also capable of measuring a concentration of the fat included in an intravascular plaque, and are used for a diagnosis of the properties (fat concentration) of plaque.

In the past, the apparatuses which measure local optical absorption characteristics inside a body tissue have been developed (see reference to PTL 1 and PTL 2, for instance).

The apparatus disclosed in PTL 1 illuminates a body tissue with light having a specified wavelength, and obtains optical absorption in each part of the body tissue by obtaining a sound velocity change inside a biological body when the body tissue is illuminated and also when the body tissue is not illuminated. The apparatus is also capable of obtaining an optical absorption spectral distribution (optical absorption characteristics) in each region inside the biological body by obtaining absorptions of the lights of plural wavelengths in the same way.

Moreover, the apparatus disclosed in PTL 2 is capable of measuring optical absorption characteristics in a local region based on elastic waves caused by photo-acoustic effects that are generated based on optical energy, by illuminating the inside of a body tissue with a pulsed light and thereby instantaneously heating the body tissue.

### CITATION LIST

### Patent Literature

[PTL 1] US Patent Application Publication No. 20100043557
[PTL 2] U.S. Pat. No. 5,840,023

### SUMMARY OF INVENTION

### Technical Problem

As has been described above, the analysis apparatus heats a specimen with light having a specified wavelength and measures, as a sound velocity change or elastic wave energy, a difference in an amount of temperature increase due to a difference in the optical absorption characteristics among the respective parts of the specimen. With such analysis apparatus, however, it is a problem that the accuracy in the measurement of a state of a specimen gets lower because a relationship between an optical absorption inside the specimen and a physical quantity (an amount of change in sound velocity or elastic wave energy) to be evaluated varies.

The present invention is conceived in view of the above-described problem and has an object to provide an analysis apparatus capable of analyzing a state of a specimen with high accuracy.

### Solution to Problem

In order to achieve the object described above, an analysis apparatus according to an aspect of the present invention is an analysis apparatus which analyzes a state of a specimen, and includes: a temperature adjustment unit which lowers a temperature of the specimen by cooling the specimen; a light source which heats at least a part of the specimen cooled by the temperature adjustment unit, by illuminating the specimen with light; a first temperature measurement unit which measures a change in the temperature of the specimen caused by the heating by the light source; and an analysis unit which analyzes the state of the specimen based on the temperature change of the specimen.

It should be noted that these general and concrete aspects may be realized as a system, a method, an integration circuit, a computer program, or a recording medium such as a computer-readable CD-ROM, and may also be realized through an arbitrary combination of a system, a method, an integrated circuit, a computer program, and a recording medium.

### Advantageous Effects of Invention

According to the present invention, by enhancing the relationship between an optical absorption and physical information to be measured, it is possible to provide an analysis apparatus that is capable of measuring, with higher accuracy, a distribution of optical absorption inside a specimen and a concentration distribution of an intended component.

### BRIEF DESCRIPTION OF DRAWINGS

FIG. 1A is a diagram showing a first example of an overall configuration of an analysis apparatus according to Embodiment 1.

FIG. 1B is a functional block diagram of the analysis apparatus according to Embodiment 1.

FIG. 1C is a flowchart showing the operation of the analysis apparatus according to Embodiment 1.

FIG. 2 is a diagram showing a second example of an overall configuration of the analysis apparatus according to Embodiment 1.

FIG. 3 is a diagram showing a third example of an overall configuration of the analysis apparatus according to Embodiment 1.

FIG. 4 is a diagram showing a fourth example of an overall configuration of the analysis apparatus according to Embodiment 1.

FIG. 5 is a diagram showing a fifth example of an overall configuration of the analysis apparatus according to Embodiment 1.

FIG. 6 is a diagram showing a first example of an overall configuration of a light illumination apparatus according to Embodiment 2.

FIG. 7 is a diagram showing a second example of an overall configuration of the light illumination apparatus according to Embodiment 2.

FIG. 8 is a diagram showing an example of an overall configuration of an analysis apparatus according to Embodiment 3.

FIG. 9 is a diagram showing an example of an overall configuration of an analysis apparatus according to Embodiment 4.

FIG. 10 is a diagram showing an example of an overall configuration of an analysis apparatus according to Embodiment 5.

FIG. 11 is a diagram for illustrating a problem in temperature change of a specimen.

FIG. 12 is a diagram showing an example of the temperature change of a specimen, using the analysis apparatus according to Embodiment 5.

FIG. 13 is a diagram showing another example of the temperature change in a specimen, using the analysis apparatus according to Embodiment 5.

FIG. 14 is a diagram showing an overall configuration of a conventional spectroscopic measurement apparatus.

FIG. 15 is a diagram showing an overall configuration of another conventional spectroscopic measurement apparatus.

### DESCRIPTION OF EMBODIMENTS

### Knowledge Based on which the Present Invention has been Conceived

The inventors of the present invention have discovered that the following problems rise with regard to the spectroscopic measurement apparatuses described in the Background Art, and others.

Spectroscopic measurement apparatuses which measure optical absorption characteristics inside a body tissue are capable of measuring concentration distributions of various constituents utilizing optical absorption characteristics (a relationship between a wavelength of light and an optical absorption) that vary from material to material, and are used for medical diagnosis in various fields. For example, the spectroscopic measurement apparatuses are capable of measuring concentration distributions of oxygenated hemoglobin and deoxygenated hemoglobin inside the body and determining on a formation of a new blood vessel and on an oxygen saturation of hemoglobin which are associated with tumor growth, and are utilized for diagnosis. Such spectroscopic measurement apparatuses are also capable of measuring a concentration of the fat included in an intravascular plaque, and are used for a diagnosis of the properties (fat concentration) of plaque.

With the apparatus as described above, a far-red light having a wavelength of about 600 nm to 1500 nm and high permeability characteristics with regard to a body tissue is utilized. However, the light that has permeated a body tissue propagates while repeating a strong scattering through the cells that are in the size of a few dozen μm constituting a biological body, and thus becomes a multiply scattered light (diffusion light). All the paths through which the diffusion light has traveled are not identified; therefore, it is almost impossible to obtain local optical absorption characteristics inside the body tissue.

In the past, the apparatuses which measure local optical absorption characteristics inside a body tissue have been developed (see reference to PTL 1 and PTL 2, for instance).

The apparatus disclosed in PTL 1 illuminates a body tissue with light having a specified wavelength, and obtains optical absorption in each part of the body tissue by obtaining a sound velocity change inside a biological body when the body tissue is illuminated and also when the body tissue is not illuminated. The apparatus is also capable of obtaining an optical absorption spectral distribution (optical absorption characteristics) in each region inside the biological body by obtaining absorptions of the lights of plural wavelengths in the same way.

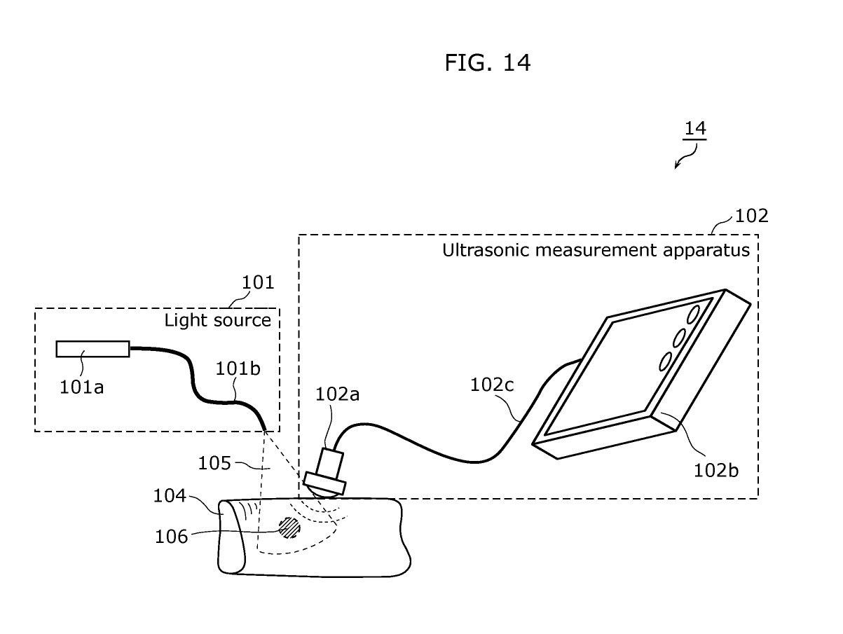

FIG. 14 is a schematic view of the ultrasonic measurement apparatus (spectroscopic measurement apparatus) as disclosed in PTL 1, utilizing the spectroscopic characteristics.

The spectroscopic measurement apparatus shown in FIG. 14 includes a light source 101 and an ultrasonic measurement apparatus 102. The following describes an operation of the spectroscopic measurement apparatus.

(1) [Process of ultrasonic sound velocity measurement (first time)]: measure an internal structure of a biological body 104 using the ultrasonic measurement apparatus 102. The ultrasonic measurement apparatus 102 includes an ultrasonic probe 102a, a main body 102b, and a cable 102c for connecting the two. The main body 102b transmits an electrical signal for vibrating the ultrasonic probe 102a via the cable 102c. The biological body 104 is illuminated with an ultrasonic pulse generated in the ultrasonic probe 102a, and the ultrasonic pulse reflected in each part inside the biological body 104 is converted back to an electrical signal in the ultrasonic probe 102a and then transmitted to the main body 102b of the measurement apparatus. (The reflection of the ultrasonic pulse occurs in the boundaries where a density or a sound velocity differs.) The main body 102b of the measurement apparatus memorizes the electrical signal from the ultrasonic probe 102a.

(2) [Start the process of selective optical heating]: the biological body 104 is illuminated with a selective heating light 105 using the light source 101 which includes a laser light source 101a and an optical fiber 101b that guides a laser light generated by the laser light source 101a to the biological body.

(3) [Process of ultrasonic sound velocity measurement (second time)]: measure again the internal structure of the biological body 104 using the ultrasonic measurement apparatus 102, as in the process (1).

(4) [Process of sound velocity change calculation]: compare an ultrasonic pulse waveform (electrical signal) reflected from the biological body 104, which is obtained in the process (1), with the one reflected from the biological body 104, which is obtained in the process (3), and obtain an amount of change in sound velocity in the respective parts inside the biological body 104 before and after the process (2).

Here, light having the most appropriate wavelength is selected for the selective heating light 105 depending on the intended use.

For example, in the case of measuring a concentration of the fat included in an intravascular plaque 106, a selective heating light having a wavelength of about 1200 nm and high fat absorption is used.

The higher the fat concentration is, the higher the absorption of the selective heating light gets and the greater the change in sound velocity becomes. Therefore, it is possible to obtain a fat concentration distribution by comparing the sound velocities before and after the illumination of the plaque 106 with the selective heating light.

With the apparatus disclosed in PTL 2, it is possible to measure the optical absorption characteristics in a local region based on the elastic waves generated by the photo-acoustic effects that are generated based on light energy, by illuminating the inside of a body tissue with a pulsed light and thereby instantaneously heating the body tissue.

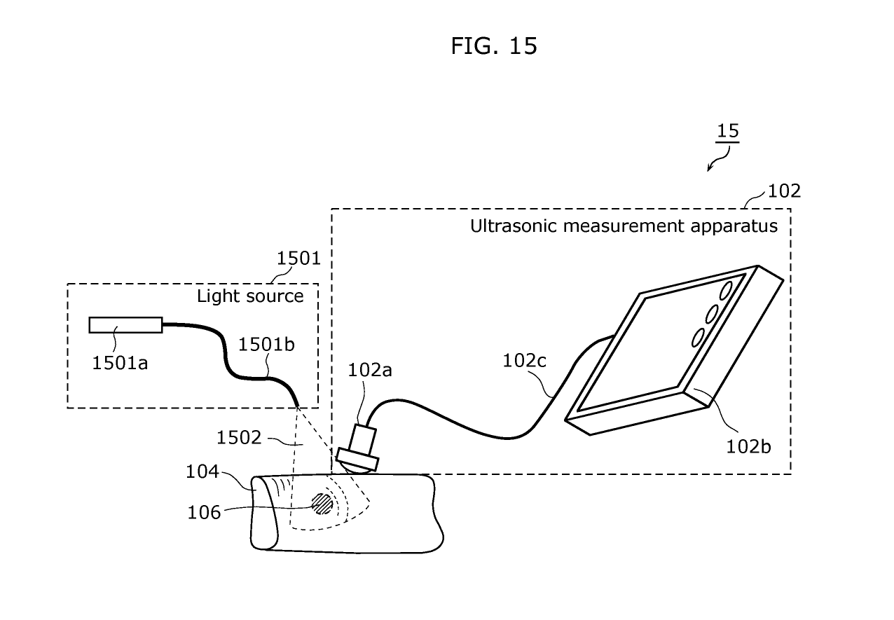

The apparatus disclosed in PTL 2 includes a pulsed light source 1501 and the ultrasonic measurement apparatus 102, as shown in the schematic view in FIG. 15.

The following describes the operation of the apparatus.

(1) [Process of generating photo-acoustic effects]: the biological body 104 is illuminated with a differential heating pulsed light 1503 using a pulsed light source 1501 that includes a pulsed laser light source 1501a, and an optical fiber 1501b that guides the pulsed laser light generated in the pulsed laser light source 1501a to the biological body. With this, instantaneous heat generation occurs in the area where the optical absorption with regard to the differential heating pulsed light 1503 is high, and instantaneous expansion associated with the rise in temperature generates ultrasonic waves (elastic waves).

(2) [Process of measuring photo-acoustic effects]: the ultrasonic measurement apparatus 102 which includes the ultrasonic probe 102a receives photo-acoustic effects inside the biological body 104, and by obtaining a location where the photo-acoustic effects have been generated and the generated energy, the part where optical absorption is high inside the biological body is obtained.

As is the case described in PTL 1 (the case of FIG. 14), in the case of using a pulsed light having a wavelength of 1200 nm, for instance, as the differential heating pulsed light 1502, it is possible to obtain a location and a fat concentration of the intravascular plaque 106 having a high fat concentration, based on the elastic wave energy and the location where the energy has been generated.

Moreover, extended applications of the above-described spectroscopic measurement apparatus (e.g., gas component analysis and examination of foreign substances contained in food) are possible, not limiting its application to a biological body. In addition, other than the examples of using ultrasonic waves, as shown in PTL 1 and PTL 2, the examples of measuring a rise in temperature caused by heating generated with light, using a thermoelectric couple, a radiation thermometer, or the like are also under consideration.

However, with the use of the analysis apparatus which heats a specimen using light having a specified wavelength and measures, as a sound velocity or elastic wave energy, a difference in an amount of temperature increase due to a difference in the optical absorption characteristics among the respective parts of the specimen, it has been a problem that the accuracy in the measurement of a state of a specimen gets lower because a relationship between an optical absorption inside the specimen and a physical quantity (an amount of change in sound velocity or elastic wave energy) to be evaluated varies.

The present invention is conceived in view of the above-described problem, and has an object to provide an analysis apparatus capable of analyzing a state of a specimen with high accuracy.

In order to achieve the object, the analysis apparatus according to an aspect of the present invention is an analysis apparatus which analyzes a state of a specimen, and includes: a temperature adjustment unit which lowers a temperature of the specimen by cooling the specimen; a light source which heats at least a part of the specimen cooled by the temperature adjustment unit, by illuminating the specimen with light; a first temperature measurement unit which measures a change in the temperature of the specimen caused by the heating by the light source; and an analysis unit which analyzes the state of the specimen based on the temperature change of the specimen.

With such configuration, a temperature distribution of a specimen is made almost homogenous by cooling the specimen, and while keeping that state, it is possible to locally heat a part of the specimen using a light source. Thus, it is possible to analyze a state of the part based on a difference between a temperature of the part when it is heated and a temperature of the part when it is not heated. In other words, the temperature distribution of the specimen is made homogenous by cooling the specimen, and the temperature increase due to the heating can be obtained with high accuracy. Moreover, the temperature of the specimen before heating has been decreased, and thus the temperature increase due to the heating can be made greater. Furthermore, the cooling of the specimen can restrain the blood flow inside the specimen and reduce a transfer of the amount of heat inside the specimen. As a result, detailed information indicating a state of the part can be obtained. Thus, it is possible to analyze a state of the specimen with high accuracy.

For example, the first temperature measurement unit includes: an ultrasonic probe which transmits an ultrasonic pulse to the specimen and receives a reflected wave that is the ultrasonic pulse reflected from the specimen; and an ultrasonic wave analysis unit which measures the temperature of the specimen based on a signal of the reflected wave received by the ultrasonic probe. The analysis apparatus further includes a storage unit which stores, into a memory unit, the signal of the reflected wave received by the ultrasonic probe, and the ultrasonic wave analysis unit measures the temperature of the specimen based on the signal of the reflected wave stored in the memory unit.

With such configuration, it is possible to measure a temperature of a specimen using the characteristics of reflected waves of ultrasonic waves. In addition, by measuring the temperature of the specimen based on the reflected waves of the ultrasonic waves that have been received for several times, it is possible to analyze a state of the specimen with higher accuracy.

For example, the first temperature measurement unit includes an ultrasonic probe which receives an ultrasonic pulse generated by the specimen, when the light source heats the specimen, and the analysis unit analyzes the state of the specimen based on the temperature change of the specimen and an intensity of the ultrasonic pulse received by the ultrasonic probe.

With such configuration, an ultrasonic probe receives the ultrasonic waves generated by a specimen in association with the heating of the part that is heated through the illumination with light. The intensity of the ultrasonic waves varies depending on the state of the part. Thus, it is possible to analyze a state of the specimen with higher accuracy by using not only the temperature change of the specimen, but also the information obtained from the ultrasonic waves generated by the specimen in association with heating.

The first temperature measurement unit is a radiation thermometer, for instance.

With this, it is possible to obtain a state of a specimen by obtaining a temperature of the specimen without contacting the specimen.

For example, the temperature adjustment unit includes: a heat absorption unit which is disposed in a position contacting the specimen and absorbs an amount of heat from the specimen; a heat exchange unit which is disposed in contact with the heat absorption unit and includes a Peltier element; a drive electric source which provides the heat exchange unit with a drive power for driving the heat exchange unit; and a heat release unit which is disposed in contact with the heat exchange unit and includes a fin that releases an amount of heat absorbed by the heat exchange unit from the specimen.

With this configuration, it is possible to effectively cool a specimen. By effectively cooling the specimen, it is possible to analyze a state of the specimen with higher accuracy.

For example, the temperature adjustment unit includes a heat absorption unit which is disposed in a position contacting the specimen at a surface that is close to the light source, comprises a material that transmits the light, and absorbs an amount of heat from the specimen, and the light source illuminates the specimen with light via the heat absorption unit.

With this configuration, by absorbing an amount of heat from the part having been heated through the illumination with light, it is possible to reduce an amount of the heat that transfers from the heated part to its periphery. Thus, it is possible to reduce the temperature increase in the parts other than the heated part and to analyze a state of a specimen with higher accuracy.

For example, the specimen is a biological body, and the analysis apparatus includes a second temperature measurement unit which measures a temperature of the heat absorption unit, and the temperature adjustment unit further adjusts the drive power based on the temperature of the heat absorption unit measured by the second temperature measurement unit, to bring the temperature of the heat absorption unit within a range of −4 to 30 degrees Celsius.

With this configuration, it is possible to analyze a state of a biological body as a specimen. Through the measurement within the range of temperature as described above, it is possible to make the biological body less affected and to obtain a state of the biological body with accuracy.

For example, the light source illuminates the specimen with light that includes plural wavelength components having wavelengths that are different from one another.

With this, it is possible to obtain a state of a biological body through plural perspectives corresponding to the wavelengths of light.

For example, the light source illuminates the specimen with a continuous wave laser (CW) light and a short pulsed light at different timings, the short pulsed light having a pulse width of 0.2 nanoseconds or longer and 330 nanoseconds or shorter.

With this, it is possible to obtain a state of a specimen based on both the information obtained from the temperature increase due to the illumination with light and the information obtained from the ultrasonic waves that the specimen has generated through the illumination with light.

For example, the analysis apparatus further includes a multi-mode fiber which guides the light generated by the light source, and the multi-mode fiber includes a winding part that is as long as one lap or longer in a part of the multi-mode fiber.

With this configuration, a specimen is illuminated with a homogenous light generated by a light source. As a result, the temperature of the part illuminated with the light rises uniformly. Thus, it is possible to analyze a state of the specimen with higher accuracy.

For example, the analysis apparatus further includes a sound velocity heat changing member which is disposed between the ultrasonic probe and the specimen and has an acoustic impedance of (1.0 to 1.4)×10⁶ kg/m²s or (1.6 to 2.25)×10⁶ kg/m²s.

With this configuration, by measuring the time necessary for an ultrasonic wave pulse to pass a sound velocity heat changing member, a temperature of a specimen can be obtained.

For example, the first temperature measurement unit includes: an optical fiber that includes a fiber grating; and a reflection characteristic measurement unit which measures the temperature of the specimen by measuring at least one of a peak reflected wavelength of the fiber grating and a reflectance of a predetermined wavelength, as a reflection characteristic.

With this configuration, by monitoring a wavelength of the light which reflects an optical fiber that includes a fiber grating, it is possible to obtain a temperature of a specimen.

The analysis apparatus further includes: a tank which stores water that includes an antiseptic agent and is used for cooling the specimen, and the temperature adjustment unit further adjusts a temperature of the water in the tank.

With this configuration, it is possible to uniformly cool a specimen with the water stored in a tank.

For example, the ultrasonic probe includes a piezoelectric body including crystal, lithium niobate, or lithium tantalate.

With this configuration, by using an ultrasonic probe for which crystal that is a transparent piezoelectric material and bulk-type transparent piezoelectric materials such as lithium niobate and lithium tantalate are used, it is further possible to place a heat absorption unit in the part that is heated by the illumination with light. As a result, the amount of heat that transfers from the heated part to its periphery can be reduced. Thus, it is possible to restrain the temperature increase in the parts other than the heated part and to analyze a state of a specimen with higher accuracy.

For example, the specimen is a biological body, the light source illuminates the specimen with light having a wavelength of 1100 nm or longer and 1300 nm or shorter, and the analysis unit measures fat concentration of a predetermined portion inside the biological body as the state of the specimen.

With this, by illuminating a biological body with the light having a wavelength that a fat in the biological body can easily absorb, a fat concentration of the biological body can be obtained as a state of a specimen.

For example, the temperature adjustment unit further increases the temperature of the specimen by heating the specimen.

With this, it is possible to make the temperature distribution of a specimen homogenous by uniformly heating the specimen.

For example, the temperature adjustment unit includes a microwave source which heats the specimen by illuminating the specimen with a microwave.

With this, it is possible to make the temperature distribution of a specimen homogenous by uniformly heating the specimen with microwaves.

For example, after the light source illuminated the specimen with the light, the ultrasonic probe transmits an ultrasonic pulse to the specimen and receives a first reflected wave that is the reflected wave, and when the light source is illuminating the specimen with the light, the ultrasonic probe transmits an ultrasonic pulse to the specimen and receives a second reflected wave that is the reflected wave, and the ultrasonic wave analysis unit measures the temperatures of the specimen based on signals of the first reflected wave and the second reflected wave as the first temperature and the second temperature, respectively.

With this, it is possible to obtain a state of a specimen with regard to the part that is heated through the illumination with light, based on the temperatures of the specimen during and after the heating. After the heating is ended, an amount of change in temperature is large because the temperature suddenly drops by the transfer of the amount of heat from the heated part to its periphery. Thus, it is possible to obtain the state of the specimen in more detail because the difference between the temperature during the heating and the temperature after the heating is large.

For example, after the light source illuminated the specimen with the light, the ultrasonic probe receives a first reflected wave and a second reflected wave which are reflected waves from the specimen and the ultrasonic wave analysis unit measures the temperatures of the specimen based on signals of the first reflected wave and the second reflected wave as the first temperature and the second temperature, respectively.

With this, it is possible to obtain a state of a specimen with regard to the part that is heated through the illumination with light, based on the temperature of the specimen that is measured at least two times after the heating is ended.

For example, the ultrasonic probe receives the second reflected wave within twenty seconds after the reception of the first reflected wave.

With this, it becomes possible to reduce the occurrence of the measurement errors due to breathing, by measuring the temperature of a specimen for two times within 20 seconds that is a time duration during which any one can stop breathing.

For example, the ultrasonic probe transmits, to the specimen, two ultrasonic pulses having waveforms that are different from each other, and receives the first reflected wave and the second reflected wave as the reflected waves of the two ultrasonic pulses.

With this, it is possible to measure a temperature of the part that is close to the surface of the specimen and a temperature of the part that is far from the surface of the specimen. Thus, it is possible to obtain a state of the specimen in more detail.

It should be noted that these general and concrete aspects may be realized as a system, a method, an integration circuit, a computer program or a recording medium such as a computer-readable CD-ROM, and may also be realized through an arbitrary combination of a system, a method, an integrated circuit, a computer program and a recording medium.

The following discusses again the causes of the problem in detail.

With the conventional spectroscopic measurement apparatus which heats a specimen with light having a specified wavelength, and measures a difference in an amount of temperature increase due to a difference in the optical absorption characteristics among the respective parts of the specimen, as a velocity change or elastic wave energy, it is a problem that accuracy in the measurement of a state of the specimen gets lower because a relationship between an optical absorption inside the specimen and a physical quantity (an amount of change in sound velocity or elastic wave energy) to be evaluated varies.

For example, the optical absorption characteristics (optical absorption) in each part are proportional to an amount of heat generation, however, a unique optical absorption and a unique amount of temperature increase cannot necessarily be identified. The amount of heat transferred from the part where heat generation is large to the part where heat generation is small varies because a heat capacity and heat conductivity vary depending on a structure and a material composition of a specimen. Namely, even if there is a part where the amount of heat generation is especially large (optical absorption is high), if the amount of heat that transfers to the periphery is large, the temperature difference between the heated part and its periphery is reduced.

In addition, with the use of the spectroscopic measurement apparatus which obtains optical absorption based on an amount of change in sound velocity, as disclosed in PLT1, measurement accuracy gets lower because a rate of change in the temperature of the sound velocity varies depending on the material composition of a specimen.

Furthermore, a relationship between unique elastic wave energy and a unique amount of temperature increase cannot be identified because a rate of volume expansion, a heat capacity, or a sound velocity varies depending on the material composition of a specimen.

The present invention, therefore, realizes a highly accurate analysis apparatus by the methods as described below.

(1) The analysis apparatus attempts for high accuracy in the analysis results by having a function to obtain at least one of the above-described relationships which cause the reduction in measurement accuracy.

(2) The analysis apparatus attempts for high accuracy in the analysis results by minimizing variability in at least one of the above-described relationships which cause the reduction in measurement accuracy (e.g., variability among samples, variability in locations, etc.).

The following describes an analysis apparatus according to an aspect of the present invention with reference to the drawings.

Each of the exemplary embodiments described below shows a general or specific example. The numerical values, shapes, materials, structural elements, the arrangement and connection of the structural elements, steps, the processing order of the steps etc. shown in the following exemplary embodiments are mere examples, and therefore do not limit the inventive concept, the scope of which is defined in the appended Claims and their equivalents. Therefore, among the structural elements in the following exemplary embodiments, structural elements not recited in any one of the independent claims defining the most generic part of the inventive concept are not necessarily required to overcome conventional disadvantage(s).

Moreover, same reference numerals are used for same constituent elements and their descriptions may be abbreviated in some cases.

### Embodiment 1

The present embodiment describes an example of an analysis apparatus which targets a biological body such as a human body or a body of an animal as a specimen, enhances a relationship between optical absorption and an amount of temperature increase by reducing a heat transfer caused by blood flow, and obtains an optical absorption distribution inside the specimen with higher accuracy.

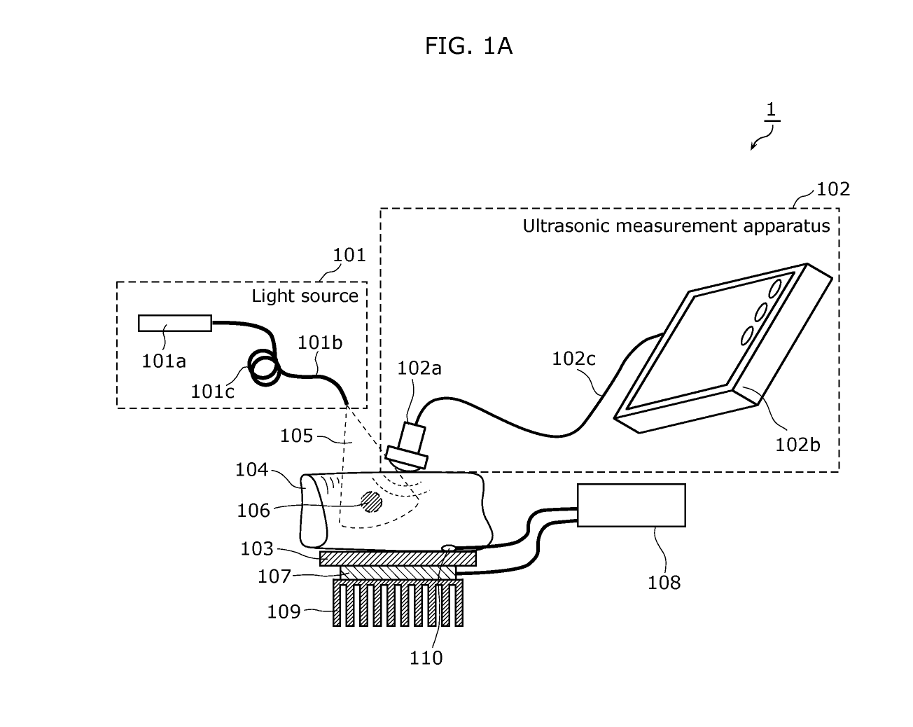

FIG. 1A is a diagram showing the first example of an overall configuration of an analysis apparatus 1 according to the present embodiment. FIG. 1B is a functional block diagram showing the analysis apparatus according to Embodiment 1.

As shown in FIG. 1A, the analysis apparatus 1 includes the light source 101, the ultrasonic measurement apparatus 102, and a specimen contact part 103.

As shown in FIG. 1B, the analysis apparatus 1 includes, as a functional block, a light source 1a, a first temperature measurement unit 1b, a temperature adjustment unit 1c, an analysis unit 1d, and a storage unit 1e.

The light source 1a heats at least a part of the specimen that has been cooled by the temperature adjustment unit 1c, by illuminating the specimen with light. The light source 1a is an equivalent of the light source 101 in FIG. 1A.

The first temperature measurement unit 1b measures a change in the temperature of the specimen, which is caused by the heating with the light source 1a. The first temperature measurement unit 1b is an equivalent of the ultrasonic measurement apparatus 102 in FIG. 1A.

The temperature adjustment unit is lowers the temperature of the specimen by cooling the specimen. The temperature adjustment unit 1c is an equivalent of the specimen contact part 103 in FIG. 1A.

The analysis unit 1d analyzes a state of the specimen based on the temperature change of the specimen.

The storage unit 1e stores a signal received by the ultrasonic measurement apparatus 102 into a memory unit (not shown in the diagram).

FIG. 1C is a flowchart showing an operation of the analysis apparatus 1 according to the present embodiment.

(1) [Start the process of cooling of biological body (S101)]: the analysis apparatus 1 lowers the temperature of the biological body 104 by allowing the specimen contact part 103, which has the temperature lower than the biological body, to contact the biological body 104.

(2) [Process of ultrasonic sound velocity measurement (first time) (S102)]

(3) [(Start) Process of selective optical heating (S103)]

(4) [Process of ultrasonic sound velocity measurement (second time) (S104)]

(5) [Process of calculation of sound velocity change (S105)]

The process of cooling a biological body will be described in detail. The analysis apparatus 1 according to the present embodiment firstly cools a biological body that is a specimen, and reduces a heat transfer caused by blood flow.

After the biological body has been cooled to a satisfactory extent, the analysis apparatus 1 measures sound velocities inside the specimen when the biological body is illuminated with a selective heating light and when the biological body is not illuminated with such light. Next, the analysis apparatus 1 compares the measured sound velocities, and by obtaining optical absorption in each part of the specimen based on the sound velocity caused by the light illumination, it is possible to measure a distribution of component concentration.

The below describes the analysis apparatus 1 which illuminates the biological body 104 with a selective heating light 105 having a wavelength in the range of 1100 nm or longer and 1300 nm or shorter, or more preferably, having a wavelength of 1200 nm, and measures an amount of fat (fat concentration) of a plaque inside a blood vessel (intravascular plaque) 106, as in the conventional case.

In a fat tissue, light having a wavelength of 1200 nm or so has a high optical absorption. The part where a fat concentration is high inside the biological body 104 greatly absorbs the light having a wavelength of 1200 nm or so and indicates a temperature increase that is greater than the part where the fat concentration is low. A propagation velocity of a sound wave including an ultrasonic wave varies depending on a change in the temperature of a medium. As has been described above, the analysis apparatus 1 thus becomes capable of obtaining optical absorption based on a sound velocity inside the biological body 104 by comparing an ultrasonic pulsed signal received by the ultrasonic probe 102a at the time when the biological body 104 is illuminated with the selective heating light and the one received at the time when the biological body 104 is not illuminated with such light, and thus, becomes capable of obtaining a fat concentration.

With the conventional spectroscopic measurement apparatus 14 as shown in FIG. 14, the heat generated in the intravascular plaque 106 where an optical absorption of the selective heating light 105 is especially high is transferred to the periphery due to blood flow. As a result, an amount of temperature increase (an amount of change in sound velocity) of the plaque 106 varies depending on an amount of the blood flow, and it has been difficult to obtain an accurate optical absorption.

To overcome this difficulty, the analysis apparatus 1 according to the present embodiment restrains an amount of blood flow by cooling a biological body. The control of the heat transfer caused by the blood flow minimizes the variability in a relationship between optical absorption and an amount of temperature increase (proportionality coefficient).

Namely, it is possible to provide an analysis apparatus capable of measuring a distribution of optical absorption and also a component concentration both with accuracy higher than that achieved with the conventional configuration.

The following describes in detail a configuration of the analysis apparatus 1 according to the present embodiment.

First, an optical fiber is used as a means to guide, to the biological body, a laser light beamed from the laser light source 101a; however, an optical system using a lens or a mirror may be used instead. It is especially desirable to use an optical fiber because it realizes a more compact and lightweight light guiding means.

As for the light source 101, a light source that generates light having a specified wavelength, such as an LED and a lamp equipped with a wavelength filter, can be used other than a laser light source. In the case of using an optical fiber as a light guiding means, however, it is desirable to use a laser light source as a light source. With such use, an analysis apparatus that enables lower consumption power can be realized.

Moreover, it is desirable to use a multi-mode fiber for an optical fiber. Furthermore, it is also preferable that an optical fiber includes a winding part 101c that is as long as at least one lap or longer. This enables more homogenous illumination with light; therefore, an analysis apparatus capable of measuring, with higher accuracy, a distribution of a component inside the biological body can be realized.

It is also desirable that the specimen contact part 103 is made of a material made up of a metal such as iron, aluminum, and copper, or is made of a material such as diamond and graphite that has high heat conductivity. With this, it is possible to lower the temperature of the biological body 104 with higher speed. This enables the analysis apparatus to improve the measurement speed; therefore, it is preferable to use the above-described materials for the specimen contact part 103.

It is further desirable that the specimen contact part 103 has a concavo-convex shape to fit the biological body, and this enlarges the area contacting the biological body 104. This enables measurement with higher speed.

The temperature adjustment unit may include, in addition to the specimen contact part 103, a heat exchange unit 107 such as a Peltier element or a compressor which absorbs heat from the specimen contact part 103, a driving electric source 108 which drives the heat exchange unit 107, and a heat release unit 109 which releases the heat absorbed from the specimen contact part 103 by the heat exchange unit 107.

In the case where the temperature adjustment unit does not include the heat exchange unit 107, and the specimen contact part 103 having a large heat capacity is used, greater cooling effects can be gained without any of the units such as the heat exchange unit, the driving electric source, and the heat release unit. This is a desirable configuration in that a more inexpensive analysis apparatus can be realized.

It is preferable to cause the heat exchange unit 107 driven by the driving electric source 108 to transfer the heat of the specimen contact part 103 to the heat release unit 109 that is a fin or a combination of a fin and a fan because this realizes a more lightweight and highly accurate analysis apparatus.

It is desirable to place a temperature measurement unit 110 (also referred to as “the first temperature measurement unit”) such as a thermistor on the specimen contact part, and to control the driving electric source 108 using the information related to the temperature of the specimen contact part measured by the temperature measurement unit 110. This renders it possible to set the temperature of the biological body 104 to be a temperature that is more appropriate for measurement, and also to minimize the variability among the temperatures of the biological body 104 obtained in the respective measurements. This enables highly reproducible measurement.

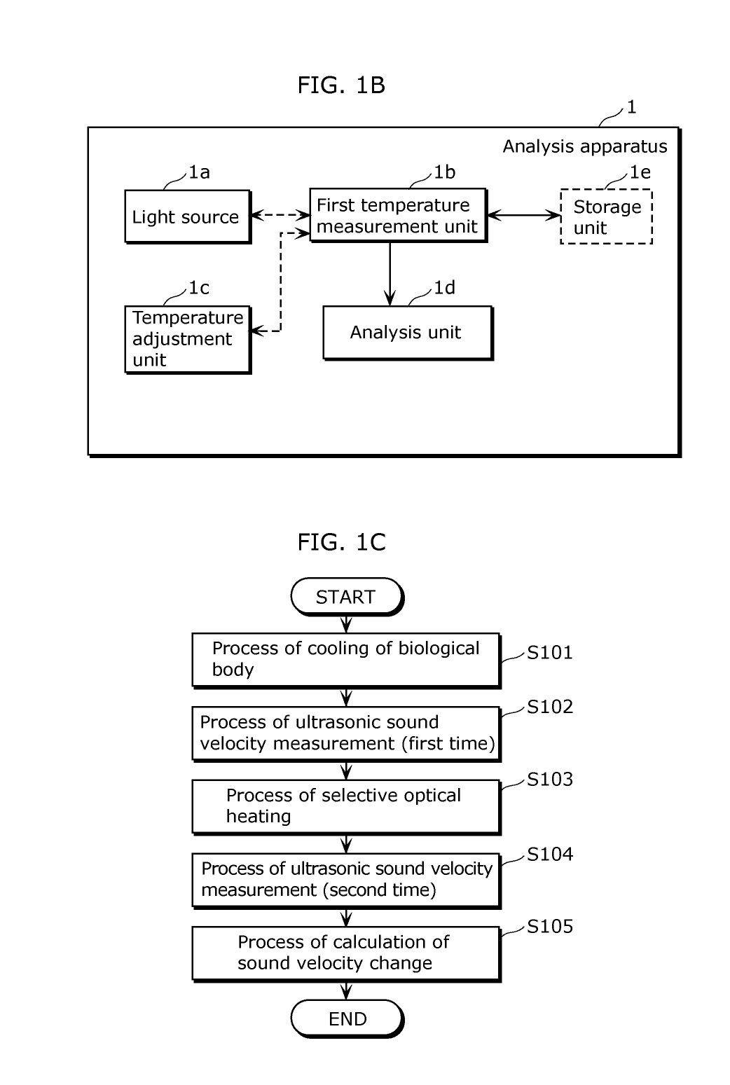

As shown in FIG. 2, the present invention may be an analysis apparatus 2 which uses a specimen contact part 201 having high light transmissibility and illuminates the biological body 104 with the selective heating light 105 through the specimen contact part 201.

The configuration of the analysis apparatus 1 in FIG. 1A is desirable in that it is possible to choose, for the specimen contact part 103, an inexpensive material such as copper and aluminum that has a high light transmissibility, because high light transmissibility is not required for the specimen contact part 103.

Whereas with the analysis apparatus 2 in FIG. 2, the temperature easily increases for its high light intensity and the heat is removed from the surface of the biological body, which is to be illuminated with the selective heating light 105 and is the part where an amount of blood flow easily increases. As a result, this enables a more homogenous reduction of the temperature inside the biological body. It is therefore possible to homogenously reduce the amount of blood flow in the whole area of the specimen from the part that is close to the surface to be illuminated with the light to the deep portion of the biological body. That is, a more comprehensive and highly accurate measurement of component concentration can be achieved.

As to the analysis apparatus 2 in FIG. 2, a material having a high thermal resistance and a high transmissibility to transmit the selective heating light 105, such as quartz and diamond, is desirable as a material of the specimen contact part 201. The diamond, in particular, has high heat conductivity and is desirable as the material of the specimen contact part according to the present embodiment.

As is the case of the analysis apparatus 1, by having the temperature measurement unit 110, a highly reproducible measurement can be achieved even with the analysis apparatus 2 in FIG. 2

In the case of cooling from the surface to be illuminated with the selective heating light, it is more desirable to use a transparent temperature measurement unit or a radiation thermometer. Thus, it is possible to measure the surface temperature of the biological body irrespective of the contact between the biological body and the specimen contact part (contact thermal resistance). In addition, another advantage is that a response speed is high.

With this configuration, it is possible to more homogenously illuminate the biological body with the selective heating light 105, which enables the measurement of component concentration distribution with higher accuracy.

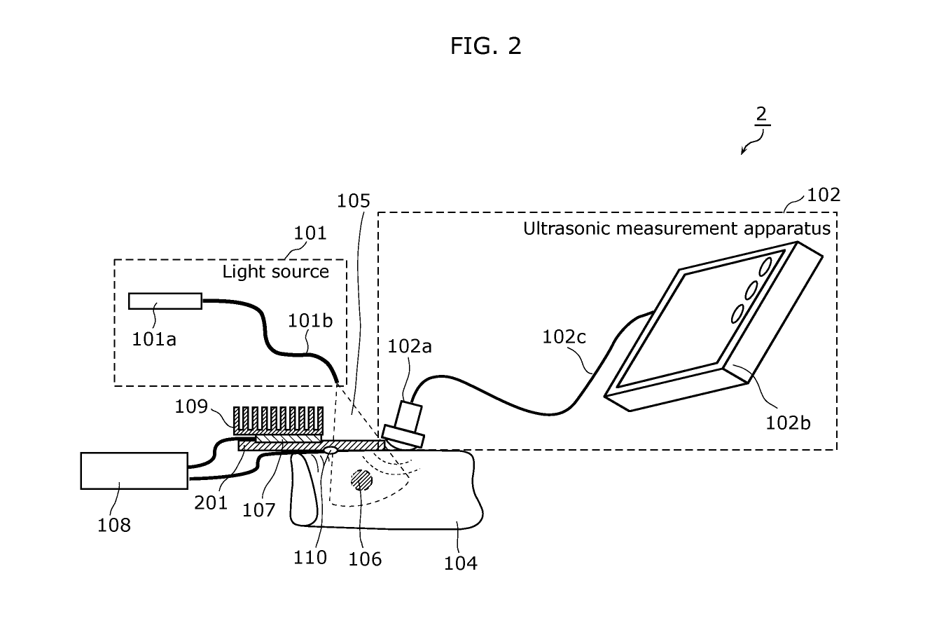

With an analysis apparatus 3 having a specimen contact part 301 inserted between the ultrasonic probe 102a and the biological body 104, as shown in FIG. 3, it is possible to more homogenously cool the inside of the biological body 104 compared to the analysis apparatus 2 in FIG. 2. Thus, it is desirable to more homogenously cool the whole area because this enables the analysis apparatus to measure with high accuracy.

As is the same as the analysis apparatus 1 in FIG. 1A, by having a temperature measurement unit such as a thermistor, the analysis apparatus 3 in FIG. 3 can achieve a highly reproducible measurement.

However, with the analysis apparatus 3 in FIG. 3, it is more desirable to set a sound velocity heat changing member 302 with which a sound velocity changes by temperature change, in a location where an ultrasonic pulse radiated from the ultrasonic probe 102a passes.

With this configuration, merely by measuring the time at which an ultrasonic pulse passes the sound velocity heat changing member 302, using the ultrasonic measurement apparatus 102, it is possible to obtain the temperature of the specimen contact part 301.

For the sound velocity heat changing member 302, it is desirable to use a material with which a sound velocity greatly changes due to temperature change. For example, materials such as rubber and resin can be used as the material of the sound velocity heat changing member 302. These materials are desirable because a low-cost and lightweight ultrasonic probe can be achieved.

Moreover, it is also desirable to use a material having a glass-transition point that is close to a normal temperature for the material of the sound velocity heat changing member 302. This is because a sound velocity change due to temperature change is large, and a highly accurate measurement can be achieved.

It should be noted that in the case of having the sound velocity heat changing member between a biological body and an ultrasonic probe, it is desirable to use a material having acoustic impedance which is different from those of the biological body and the ultrasonic probe. It is especially desirable to use a material having acoustic impedance of 1.4×10⁶ kg/m²s or smaller, or 1.6×10⁶ kg/m²s or greater.

This generates a greater reflection of the ultrasonic pulse in the boundary surface between the sound velocity heat changing member and the biological body and in the boundary surface between the sound velocity heat changing member and the ultrasonic probe. Thus it is possible to measure the temperature with high accuracy.

It is also desirable to reduce the reflection to a certain extent or smaller. For this, it is desirable that the sound velocity heat changing member has acoustic impedance of (1.0 to 1.4)×10⁶ kg/m²s or (1.6 to 2.25)×10⁶ kg/m²s, which realizes a more sensitive ultrasonic probe.

For example, a mixture of polyethylene, silica, and acryl can be used for the material of the sound velocity heat changing member. This is desirable because it is possible to realize a more inexpensive temperature measurement means compared to the case of using a thermistor or a radiation thermometer, and it is also possible to provide a low-cost spectroscopic measurement apparatus.

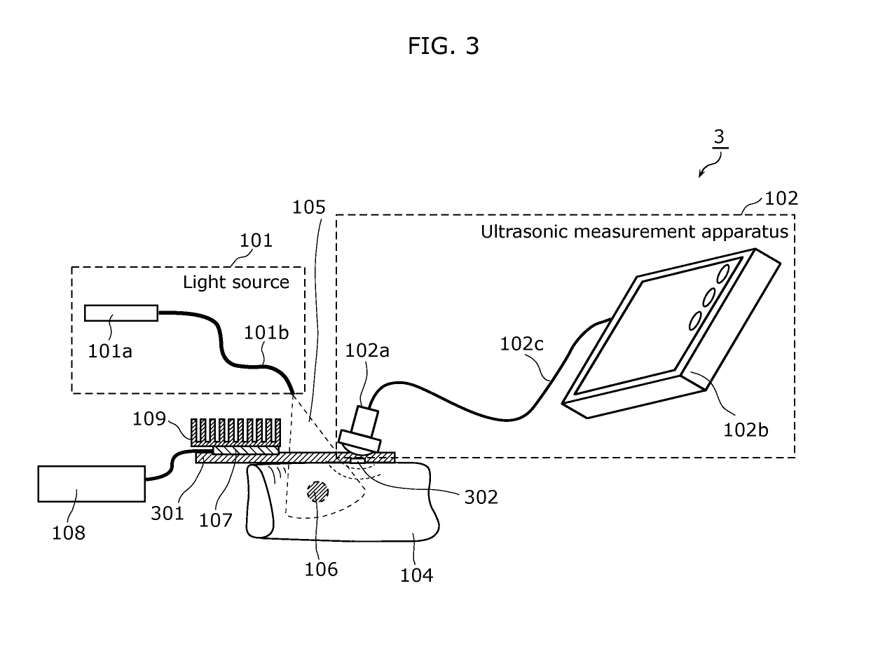

It is more desirable to have an optical fiber equipped with a fiber grating 401 within the area to be illuminated with the selective heating light 105, as shown in FIG. 4.

The fiber grating 401 can be designed in such a way that a reflection rate of the light having an arbitrary wavelength gets higher according to a grating cycle. With the change in the temperature of the fiber grating 401, a reflective indexes of the grating part changes, and thereby the wavelength of the reflecting light changes.

That is to say that the fiber grating can be used as a temperature measuring means by monitoring the wavelength of the reflecting light.

Moreover, with the use of the fiber grating 401 as a temperature measuring means, it is possible to set the temperature measuring means in the part where light and ultrasonic waves pass, which enables the adjustment of temperature with higher accuracy. Namely, this enables further minimization of the variability among measurements due to the variability among the temperatures obtained in the respective measurements.

Even with the configuration shown in FIGS. 2 through 4, it is desirable to cause the heat exchange unit 107 driven by the driving electric source 108 to transfer the heat of the specimen contact part to the heat release unit 109 that is a fin or a combination of a fan and a fin, because this can realize a more lightweight analysis apparatus.

It is especially desirable that the analysis apparatus is configured to include a heat exchange unit made up of a Peltier element, and a heat release unit made up of fin only. In such case, there being less vibration, it is possible to realize an analysis apparatus capable of measuring with high accuracy.

In addition, it is desirable to set a temperature measurement unit such as a thermistor on a specimen contact part, and to control the driving electric source 108 using the information related to the temperature of the specimen contact part measured by the temperature measurement unit. In such case, it is possible to set the temperature of the biological body 104 to a temperature that is more appropriate for the measurement, and also, to minimize the variability in the temperatures of the biological body 104 obtained in the respective measurements, which enables a highly reproducible measurement.

Furthermore, it is desirable that the analysis apparatus according to the present embodiment includes a means to monitor a driving current of the laser light source 101a and an output of the selective heating light 105. After the start of the optical heating of the biological body, it is desirable to increase the driving current provided for the heat exchange unit 107 and to enhance the cooling effects. Thus, it is possible to illuminate the biological body 104 with the selective heating light 105 of greater output and to realize a highly accurate analysis apparatus.

It is also desirable that the temperature of the specimen contact part be controlled to be −4 degrees Celsius or higher when the internal structure of the biological body is measured by the ultrasonic measurement apparatus. Thus, it is possible to prevent the skin of the specimen from getting frostbitten.

Moreover, it is more desirable that the temperature of the specimen contact part should be controlled to be 15 degrees Celsius or higher. In this case, it is possible to provide the cells with a necessary amount of oxygen. Therefore, the biological body does not easily feel the fatigue caused by a drop in the body temperature even if the measurement is performed for a long time.

It is also desirable that the temperature of the specimen contact part be controlled to be 25 degrees of Celsius or lower. In this case, it is possible to cool the biological body without being affected by an individual difference in body temperature.

In the case of illuminating the selective heating light 105 through the specimen contact part 201, as shown in FIG. 2, it is desirable that the temperature of the specimen contact part should indicate a room temperature or higher. This can prevent the generation of dew condensation on the specimen contact part, and can restrain the inhomogeneity in the illumination of the biological body 104 with the selective heating light 105, which is caused by the dew condensation. That is to say that a highly reproducible light illumination can be achieved and the variability among the accuracies in the respective measurements can be minimized.

In the case of illuminating the selective heating light 105 through the specimen contact part 201, as shown in FIG. 2, it is desirable that the temperature of the specimen contact part should indicate 30 degrees Celsius or lower. This can restrain the inhomogeneity in the illumination of the biological body 104 with the selective heating light 105, which is caused by sweating on the skin surface. It is thus possible to achieve a highly reproducible light illumination and minimize the variability among the accuracies in the respective measurements.

In order to prevent the influence caused by an individual difference in sweating temperature depending on race, sex, humidity, and others, it is desirable to adjust the temperature of the specimen contact part 201 after the measurement of a sweating temperature of a biological body being a specimen, in order that the temperature of the specimen contact part 201 does not go beyond the sweating temperature that has been measured.

With the use of an ultrasonic probe made of transparent piezoelectric material, it is possible to illuminate a biological body with both light and ultrasonic waves from the same place. By using an ultrasonic probe for which crystal that is a transparent piezoelectric material and bulk-type transparent piezoelectric materials such as lithium niobate and lithium tantalate are used, both the transmission of the ultrasonic waves by the ultrasonic probe and the light illumination on the contact surface between the ultrasonic probe and the biological body can be achieved with low cost. This is desirable because the light intensity of the biological body near the ultrasonic probe becomes more homogenous and stronger, and a more highly accurate and sensitive measurement can be achieved.

It is desirable to use a transparent piezoelectric material utilizing a single crystal thin film technology, such as zinc oxide (ZnO) and aluminum nitride (AIN), because a more compact analysis apparatus can be realized.

It is further desirable to use an ultrasonic probe which applies a voltage to a piezoelectric material using a transparent electrode such as indium tin oxide (ITO) that has excellent light transmissibility characteristics. This achieves a more sensitive and highly accurate measurement of component concentration.

Moreover, it is further desirable to use a transparent electrode made of zinc oxide series or magnesium. This achieves, with low cost, a sensitive and highly accurate measurement of component concentration.

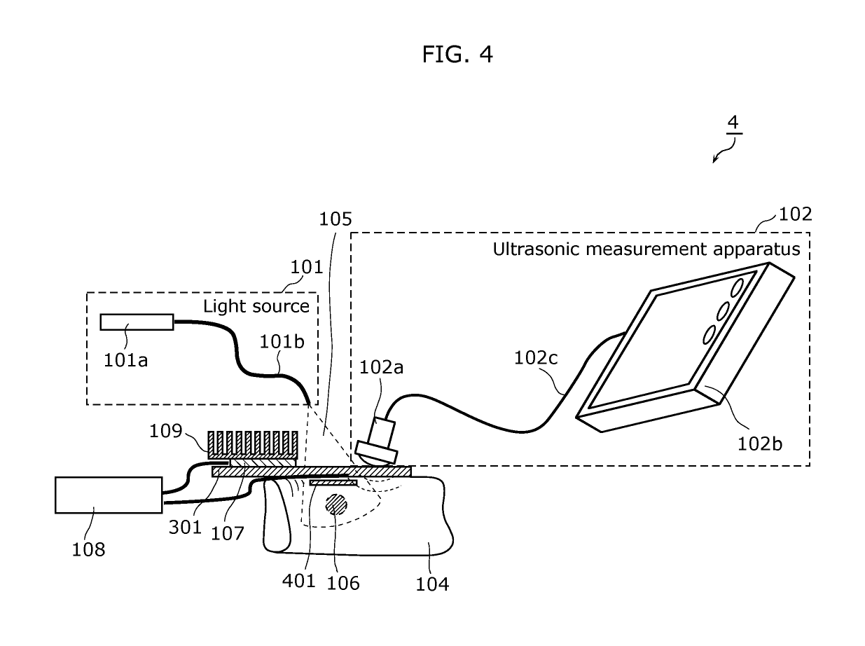

As shown in FIG. 5, the present invention may be an analysis apparatus which illuminates the biological body 104 that is set in water 502 in a tank 501 with the selective heating light 105 as well as with the ultrasonic waves from the ultrasonic probe 102a, and measures again the ultrasonic waves reflected inside the biological body 104.

With the configuration shown in FIG. 5, it is possible to cool the biological body 104 by adjusting the temperature of the water 502.

In order to adjust the temperature of the water 502, it is desirable to include the temperature adjustment unit as shown in FIG. 1A. Thus, it is possible to more freely adjust the temperature and minimize the variability among the measurements.

It is desirable to set the temperature of the water 502 to 15 degrees Celsius or higher. Thus, the amount of blood flow which enables the provision of oxygen necessary for the cells inside the biological body is maintained. Therefore, the biological body does not easily feel the fatigue due to a drop in the body temperature even if the measurement is performed for a long time.

It is also desirable to set the temperature of the water 502 to 25 degrees Celsius or lower. Thus, it is possible to cool the biological body without being affected by individual difference in body temperature.

Although it is described that the water is in the tank 501, it shall not necessarily be water. It is, however, desirable to use a liquid that is relatively runny. With such liquid having a relatively low viscosity, it is possible to effectively cool the biological body by the heat convected; therefore, a highly accurate measurement of component concentration can be achieved.

Besides water, ethanol may be used for instance. Ethanol is highly effective in sterilization and there is no need to mix any antiseptic agent. Moreover, a large amount of heat is released into the air by vaporization heat; therefore, the analysis apparatus is capable of adjusting the temperature of the specimen to a low temperature with less energy.

In the case of using water, a low-cost analysis apparatus can be realized. Water is desirable as it has a reflective index and acoustic impedance both of which are nearly the same as those of a biological body, and highly effective illumination of both light and ultrasonic waves can be achieved. It is also possible to perform the measurement without directly pressing the ultrasonic probe 102a against the biological body, and the shape of the biological body will not be deformed by directly pressing the ultrasonic probe against the biological body. Water is desirable because it is possible to draw comparison with higher accuracy even in the comparison with the results of the measurements performed in the past.

It should be noted that in the case of using water, it is desirable to use water mixed with an antiseptic agent, and a highly reproducible measurement of component concentration can be achieved.

It is more desirable to use water mixed with a surface-activating agent. The use of such water inhibits the formation of blisters on the surface of the biological body and achieves a highly accurate measurement of component concentration.

In the case of measuring the component concentration inside a biological body using the analysis apparatus according to the present embodiment, it is further desirable to perform the measurement under the following conditions.

For example, by delivering nicotine into a biological body, it is further possible to reduce an amount of blood flow inside the biological body. It is therefore desirable to perform a spectroscopic measurement on the biological body into which nicotine has been delivered, which further enables a highly accurate measurement.

In the case of delivering nicotine into the biological body through smoking or secondhand smoke, it is more desirable that the analysis apparatus performs a spectroscopic measurement within an hour and a half after the delivery of the nicotine. As it is possible to perform the measurement in the condition that the amount of blood flow is reduced by the effect of nicotine, a highly accurate measurement of component concentration can be achieved.

With the use of a nicotine patch for the delivery of nicotine, it is possible to more locally reduce the amount of blood flow. This enables a highly accurate measurement of component concentration with a small amount of intake of nicotine; therefore, it is a more recommended method to be applied to the young people such as people underage.

Moreover, a method of reducing the amount of blood flow using antiphlogistic analgetic or electrical stimulation may also be used.

The decrease in the amount of blood flow through smoking or secondhand smoke is a low-cost implementation tool; however, it is necessary to adjust the amount of blood flow to be decreased. With the method using antiphlogistic analgetic or electrical stimulation, a highly accurate measurement of component concentration can be achieved, which further enables a highly accurate measurement.

Whereas caffeine works to increase the amount of blood flow; therefore, it is more desirable that the component concentration measurement is performed within 15 minutes or 30 minutes or more after the ingestion of caffeine. This further enables a highly accurate measurement of component concentration.

It is also desirable to perform a measurement of the component concentration in a state where the part for which the component concentration is measured and the periphery of that part are pressurized. By applying additional pressure, it is possible to restrain the blood flow; therefore, it is further possible to measure component concentration with higher accuracy.

As has been described above, in the present embodiment, an analysis apparatus which obtains optical absorption based on the amount of temperature change in sound velocity has been illustrated. Even with the analysis apparatus which obtains optical absorption based on elastic wave energy, as has been described in the conventional example shown in FIG. 15, by measuring component concentration in the state where a biological body being a specimen has been cooled, it is possible to reduce the transfer of heat caused by blood flow, which enables the measurement of the component concentration with higher accuracy.

Moreover, by replacing the laser light source 101a of the analysis apparatus shown in FIGS. 1A, 2, 3, and 4 with the pulsed laser light source 301a, it is possible to realize the analysis apparatus which obtains optical absorption based on elastic wave energy, and thereby the same effects can be obtained with the same configuration.

Although it is not shown in the diagram, it is desirable to use an analysis apparatus that includes a light source capable of driving both a pulsed light and a continuous wave laser (CW) light, or to use an analysis apparatus which includes two light sources of a pulsed light source and a CW light source. For, it is possible to measure an amount of the temperature increase due to optical heating, based on both a sound velocity change and elastic wave energy, which further enables a highly accurate measurement of component concentration.

It is more preferable to use a pulsed light source that is capable of adjusting the light intensity of a pulsed light. In this case, non-linear optical absorption characteristics can be obtained as well; therefore, it is possible to measure component concentration with higher accuracy.

The present embodiment has described an example of an analysis apparatus which measures a fat concentration; however, the present embodiment is not limited to this and can be applied to all of the component concentration measurements in which a phenomenon of optical heating is put into practice. For example, with the use of the light having a wavelength of 650 nm to 800 nm, it is possible to realize the analysis apparatus which measures oxygen saturation of hemoglobin (a ratio between the concentration of oxidized hemoglobin and the concentration of deoxidized hemoglobin). Furthermore, the present embodiment is applicable for use in a judgment between cancer and benign tumor and for a diagnosis on the depth of burn injury.

In either of the cases of measuring a fat concentration and of measuring concentration and oxygen saturation of hemoglobin, it is desirable to obtain optical absorption of light having plural wavelengths using a light source that generates such light having plural wavelengths. This further enables a measurement of component concentration with high accuracy.

In the case of measuring hemoglobin oxygen saturation for the property diagnosis of cancer based on elastic wave energy, it is desirable that a pulse width of a pulsed light (full-width at half maximum output) is 0.33 μs or shorter, with which a resolution necessary for the property diagnosis of cancer can be obtained.

In the case of measuring fat concentration in an intravascular plaque based on elastic wave energy, it is desirable that a pulse width of a pulsed light is shorter than 0.07 μs, with which a resolution necessary for the property diagnosis of intravascular plaque can be obtained.

It is also desirable that a pulse width of a pulsed light is 0.2 ns or shorter. In this case, it becomes possible to generate ultrasonic waves having higher transmissibility in a biological body; therefore, a component concentration can be measured in a deeper region of the biological body.

Furthermore, the present embodiment may be applied to an analysis apparatus which targets something other than a biological body. For example, it may be applied to an examination of foreign substances contained in food, or the like.

The present embodiment has described the analysis apparatus which measures heating generated with light, using ultrasonic waves; however, the analysis apparatus according to the present invention shall not necessarily use ultrasonic waves. Even with the analysis apparatus which measures a temperature change due to optical heating using a thermocouple or a radiation thermometer, the same effects can be obtained with the same configuration as that of the present embodiment.

It is desirable to use a thermocouple because it is possible to measure component concentration with lower cost.

It is desirable to use a radiation thermometer because it is possible to measure component concentration without contacting the biological body.

As has already been described in the present embodiment, the analysis apparatus utilizing the sound velocity change due to temperature increase measures heating with light, using ultrasonic waves that is an inexpensive means having an excellent property to directly advance inside the biological body. Such analysis apparatus is desirable as it realizes the measurement of component concentration (distribution) with low cost and excellent position resolution even inside the biological body.

The analysis apparatus which measures the expansion due to temperature increase as elastic wave energy is also desirable as it is capable of more prominently detecting a difference in optical absorption (a difference in expansion rate) and realizing a low-cost and high-contrast measurement of component concentration (distribution).

The present embodiment has described the configuration in which a specimen contact part is set between an ultrasonic probe and a specimen (biological body) as shown in FIGS. 3 and 4. However, the surface itself of the ultrasonic probe at which the ultrasonic probe contacts a biological body may be configured to absorb the heat of the biological body as a specimen contact part.

### Embodiment 2

As has been shown in Embodiment 1, the present invention is effective in an analysis apparatus utilizing a phenomenon of heating; however, it is also effective in another apparatus utilizing optical heating.

The below describes an example of a light illumination apparatus (hyperthermia) intended for use in a cancer treatment in which cancer tissues are killed through heating.

First, a hyperthermia will be explained.

It is known that cancer tissues are weaker against heat compared to normal tissues, and by heating the cancer tissues at 46 degrees Celsius, for instance, the tissues are killed in a few minutes. However, at 46 degrees Celsius, a part of the normal tissues may be killed as well.

Therefore, it is desirable to selectively heating the cancer tissues only. For example, by successfully reducing the temperature to 42 degrees Celsius for the normal tissues and heating only the cancer tissues at 46 degrees Celsius, it is possible to kill only the cancer tissues without damaging any normal tissues.

The following describes a light illumination apparatus 2 which selectively increases the temperature for the cancer tissues only.

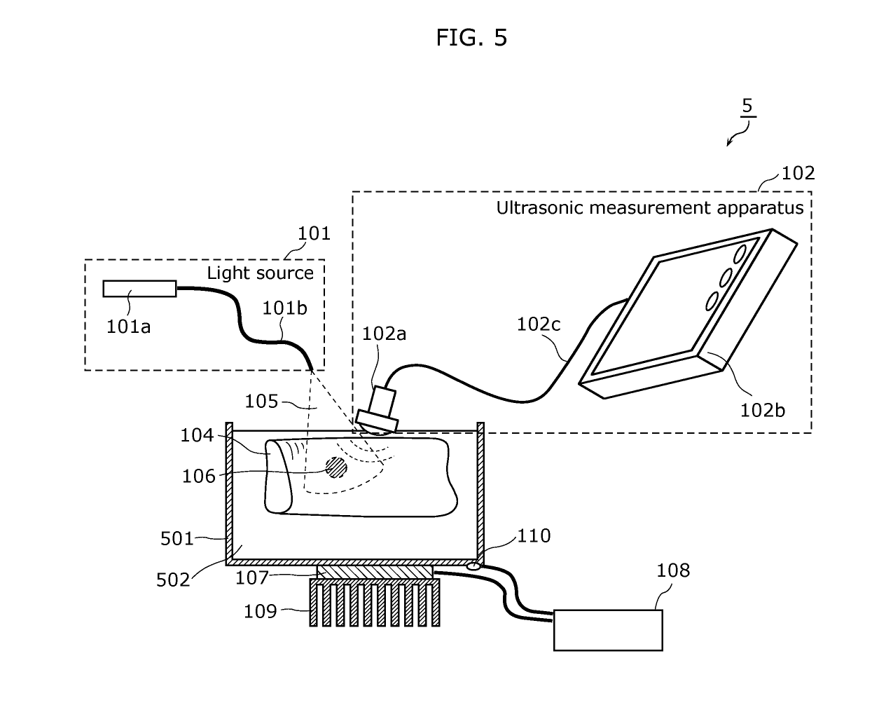

FIG. 6 is a diagram showing the first example of an overall configuration of the light illumination apparatus 6 according to the present embodiment.

As is the same as the analysis apparatus 1 in Embodiment 1, the light illumination apparatus 6 in FIG. 6 includes the light source 101, and the specimen contact part 103. As in Embodiment 1, the heat of a biological body 603 is absorbed at the specimen contact part 103, and in the state where the temperature of the body 603 is thus lowered, the biological body 603 is illuminated with a selective heating light 602 generated in the light source 101. Here, in the present embodiment, the biological body 603 is a part that has a cancer tissue 601, such as a breast or a prostatic gland.

The light source 101 generates light of which the optical absorption of cancer tissues is higher than that of normal tissues. For example, a laser light source or an LED which generates the selective heating light 602 having the wavelength of 600 nm to 800 nm is used.

With this, the selective heating light 602 can selectively heat the cancer tissue 601 inside the biological body 603.

Normally, a lot of amount of heat is transferred due to the blood flow that flows from the cancer tissue 601 towards its periphery, and this also increases the temperature of the periphery. However, with the light illumination apparatus 6 according to the present embodiment, an effect of reducing the amount of blood flow by lowering the temperature of the biological body is produced, and it is possible to more selectively increase only the temperature of the cancer tissue 601. Thus, it is possible to reduce the number of the normal tissues that are simultaneously killed when the cancer tissue 601 is killed.

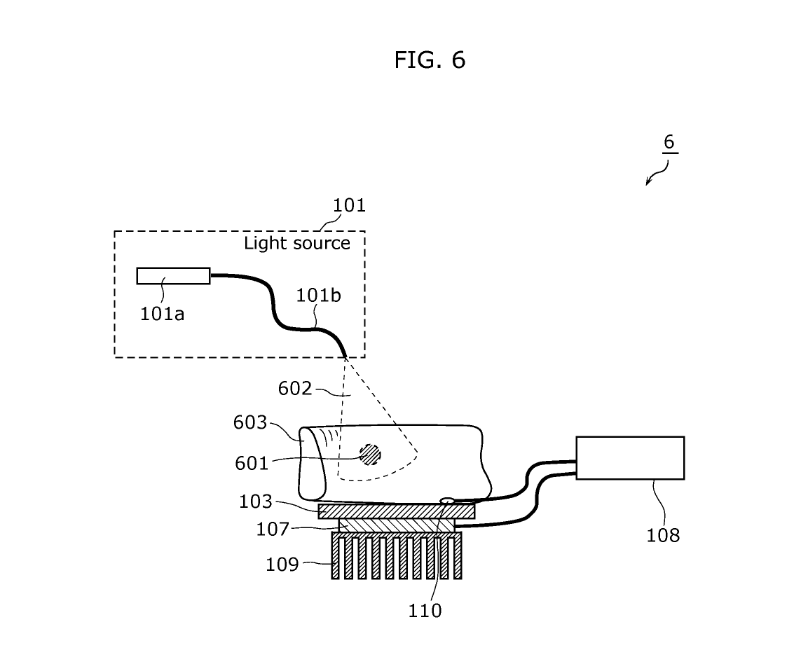

FIG. 7 is a diagram showing the second example of an overall configuration of the light illumination apparatus according to the present embodiment (light illumination apparatus 7).

The light illumination apparatus 7 in FIG. 7 is intended for use in a cancer treatment, as is the case of the light illumination apparatus 6 in FIG. 6.

The light illumination apparatus 7 in FIG. 7 includes the light source 101 and the specimen contact part 201. With the use of the light illumination apparatus 7, the heat of the biological body 603 is absorbed at the specimen contact part 201, and in the state where the temperature of the biological body 603 is thus lowered, the biological body 603 is illuminated with the selective heating light 602 generated in the light source 101.

Here, with the light illumination apparatus 7 in FIG. 7, the biological body 603 is illuminated with the selective heating light 602 through the specimen contact part 201, unlike the light illumination apparatus 6 in FIG. 6. It is therefore desirable that the specimen contact part 201 in the light illumination apparatus 7 is composed of a member that transmits the selective heating light 602. For example, by using a material such as an acrylic resin or a quartz base that ensures high light transmissibility, for the specimen contact part 201, it is possible to effectively illuminate the biological body 603 with the selective heating light 602, which lowers the consumption power.

By using a material such as diamond that is transparent and ensures high heat transmissibility, for the specimen contact part 201, it is possible to further enhance the effects of cooling the biological body 603 and thereby to reduce the amount of normal tissues that may die.

It is desirable that the specimen contact part 201 includes the heat exchange unit 107, the driving electric source 108, and the heat release unit 109, as shown in FIG. 6. This enables further reduction of the amount of normal tissues that may die.

The configuration of the light illumination apparatus 6 in FIG. 6 is desirable in that a material that is inexpensive and has a high heat transmissibility, such as aluminum and copper, can be selected for the specimen contact part 103 because high light transmissibility is not required for the specimen contact part 103.

Whereas with the light illumination apparatus 7 in FIG. 7, the heat is removed from the surface of the biological body, which is to be illuminated with light and is the part where the light intensity is high and the temperature easily increases within the biological body. It is therefore possible to make the temperature inside the biological body homogenous. This enables the reduction of the amount of blood flow in the whole area from the location near the surface to be illuminated with light to the deep part of the specimen. This configuration is especially desirable for the case of killing a cancer tissue in the vicinity of the surface to be illuminated with light. Moreover, it is also desirable because the surface on which the light is to be illuminated and the surface to be cooled by the specimen contact part 201 are on the same side, which enables the application of the light illumination apparatus to a part of the biological body that is thick (large) as a specimen.

In addition, the light illumination apparatus 7 according to the present embodiment is desirable as it has the configuration as same as that of the analysis apparatus 1 in Embodiment 1 for reducing the amount of blood flow inside the biological body, and thus, it is possible to more selectively kill a cancer tissue.

### Embodiment 3

The present embodiment describes an analysis apparatus which heats a specimen with light having a specified wavelength, and measures, as a sound velocity change, a difference in temperature increase due to a difference in optical absorption characteristics among respective parts of the specimen. The analysis apparatus aims to prevent the reduction of measurement accuracy caused by the fact that a proportionality coefficient between a temperature change and a sound velocity change varies depending on a material composition and a composition ratio of the respective parts of the specimen.

For example, a sound velocity and a proportionality coefficient between a temperature change and a sound velocity change (sound velocity temperature change coefficient) vary depending on a substance.

A velocity of a sound that travels through the water is 1483 m/s at 24 degrees Celsius, and 1530 m/s at 37 degrees Celsius. Based on this, it is derived that a sound velocity temperature change coefficient is 3.6 m/s/C. Whereas a velocity of a sound that travels in a fat tissue is 1476 m/s at 24 degrees Celsius, and 1412 m/s at 37 degrees Celsius. Based on this, it is derived that a sound velocity temperature change coefficient is −4.9 m/s/C.

With regard to various organs inside the biological body, a sound velocity and a sound velocity temperature change coefficient respectively vary according to the concentrations of water, fat, and others.

Therefore, with the analysis apparatus which illuminates a biological body with light having a wavelength of 1200 nm or so and measures a sound velocity change in each part inside the biological body using an ultrasonic probe, as in Embodiment 1, with an aim to measure a fat concentration distribution inside the biological body, for instance, even if a temperature change is proportional to a fat concentration distribution, a sound velocity change is not proportional to a temperature change. That is to say that a sound velocity temperature change coefficient may be affected in a different manner depending on the location inside the biological body according to the composition ratios of the components other than fat.

Therefore, the present embodiment describes an example of an analysis apparatus capable of measuring a desired component concentration distribution with higher accuracy, by obtaining optical absorptions with higher accuracy through the obtainment of the sound velocity temperature change coefficients inside the specimen.

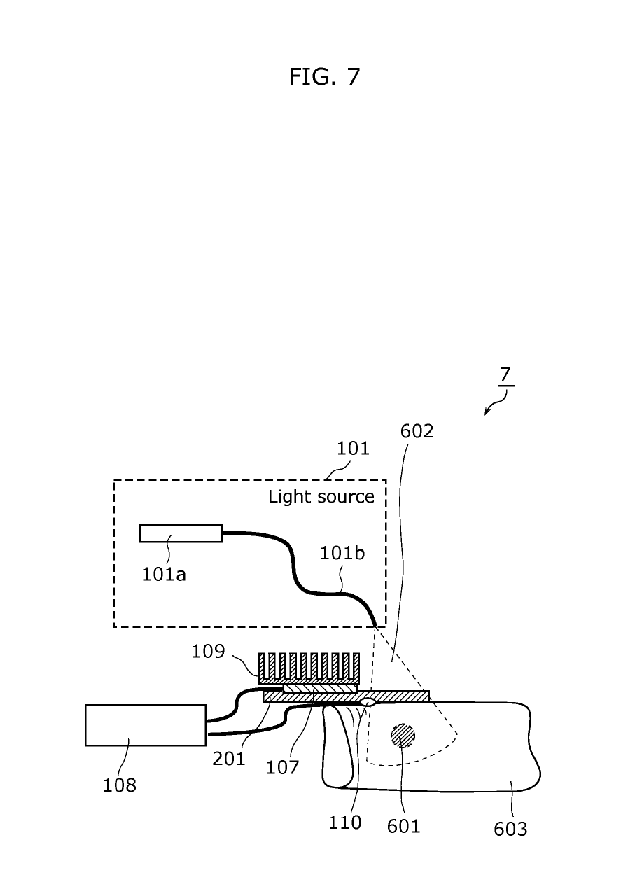

FIG. 8 is a diagram showing an example of an overall configuration of an analysis apparatus 8 according to the present embodiment.

The analysis apparatus 8 shown in FIG. 8 includes the light source 101, the ultrasonic measurement apparatus 102, a specimen contact part 801, the heat exchange unit 107, and the driving electric source 108.

It should be noted that the analysis apparatus 8 according to the present embodiment uses a measurement method that is different from the one described in Embodiment 1.

The analysis apparatus 8 according to the present embodiment performs measurement in the following procedure in the state where the biological body 104 contacts the ultrasonic probe 102a and the specimen contact part 801.

(1) [Process of ultrasonic sound velocity measurement (first time)]

(2) [Start of homogenous heating/cooling]: start heating (or cooling) the biological body 104 by the heat exchange unit 107, using the driving electric source 108.

(3) [Process of ultrasonic sound velocity measurement (second time)]

(4) [Start of selective optical heating]

(5) [Process of ultrasonic sound velocity measurement (third time)]

(6) [Calculation of sound velocity change (first time)]: compare the ultrasonic pulsed waveforms (electrical signals) having reflected from the biological body 104 which are respectively obtained in the processes (1) and (3), and obtain an amount of change in sound velocity in each part inside the biological body 104 before and after the process (2).

(7) [Calculation of sound velocity change (second time)]: compare the ultrasonic pulsed waveforms (electrical signals) having reflected from the biological body 104 which are respectively obtained in the processes (3) and (5), and obtain an amount of change in sound velocity in each part inside the biological body 104 before and after the process (2).

(8) [Calculation of amount of temperature increase]: based on the results obtained in the process (6), obtain a sound velocity temperature change coefficient in each location inside the biological body 104, and obtain a temperature increase in each location inside the biological body 104 based on the results obtained in the process (7) and the sound velocity temperature change coefficients.

The analysis apparatus 8 according to the present invention performs the measurement of the ultrasonic sound velocity for at least three times, as illustrated above.

First, as has been shown in the processes (1) through (3) and (6), the ultrasonic pulsed signals reflected from the inside of the biological body 104 are compared between the one obtained when the biological body 104 is heated and the one obtained when the biological body 104 is not heated (or the one obtained when the biological body 104 is cooled and the one obtained when the biological body 104 is not cooled), using the specimen contact part 801, the driving electric source 108, and the heat exchange unit 107. The temperature change of the biological body 104 generated by the heating (cooling) method as described above is not affected by the components or the concentrations in each part inside the biological body 104; therefore, it is possible to homogenously heat (cool) the temperature of each part inside the biological body 104.

That is to say that the sound velocity change coefficients are obtained by comparing the following: an ultrasonic sound velocity obtained in the measurement (first time) in each part inside the biological body 104 when neither homogenous heating (homogenous cooling) nor differential heating is performed; and an ultrasonic sound velocity obtained in the measurement (second time) in each part inside the biological body 104 when homogenous heating (homogenous cooling) is performed and differential heating is not performed.

Next, as has been shown in the processes (3) through (5) and (7), an ultrasonic pulsed signal reflected from the inside of the biological body 104 in the measurement (second time) when homogenous heating (homogenous cooling) and illumination with selective heating light are performed is compared with an ultrasonic pulsed signal reflected from the inside of the biological body 104 in the measurement (third time) when homogenous heating (homogenous cooling) is performed but illumination with selective heating light is not performed. The light having a specified wavelength is illuminated and a distribution indicating heat generation (temperature increase) corresponding to a desired material concentration is generated, so that a sound velocity change due to the temperature change in each part is obtained.

Thus, with a means (process) to obtain the sound velocity temperature change coefficients, the analysis apparatus 8 according to the present embodiment is capable of calculating a distribution of the amount of temperature increase, which reflects more the actual condition, based on the sound velocity change when illumination with selective heating light is performed or when such illumination is not performed. Therefore, it is possible to detect component concentration with higher accuracy.

Here, it is desirable to have a means to measure the temperature of the biological body 104, like the temperature measurement unit 110 such as a thermistor and a thermal couple. In such case, sound velocity change coefficients can be obtained with higher accuracy, and thereby it is possible to measure component concentration with higher accuracy.

By using an improved configuration such as respective locations where a biological body contacts a temperature measuring means and a heating/cooling means and materials of the respective means, as shown in Embodiment 1, the same effects can be produced even in the present embodiment.

Even in the present embodiment, it is possible to illuminate the biological body 104 with the selective heating light 105 having a wavelength of 1100 nm or longer and 1300 nm or shorter, more preferably 1200 nm, and to measure a fat concentration of the intravascular plaque 106.

Although both of the homogenous heating and the homogenous cooling produce the effects described in the present embodiment, the homogenous cooling also produces the effects gained through the control of blood flow as described in Embodiment 1; therefore, it is more preferable to perform the homogenous cooling.

The following describes in detail a configuration of the analysis apparatus 8 according to the present embodiment.

As is described in Embodiment 1, an optical fiber is used as a means to guide, to the biological body, the laser light beamed from the laser light source 101a; however, an optical system using a lens or a mirror may be used instead. It is especially desirable to use an optical fiber as it realizes a more compact and lightweight light guiding means.

As for the light source 101, a light source that generates light having a specified wavelength, such as an LED and a lamp equipped with a wavelength filter, can be used other than a laser light source. In the case of using an optical fiber as a light guiding means, it is desirable to use a laser light source as a light source. With such use, an analysis apparatus that enables lower consumption power can be realized.

Moreover, it is desirable to use a multi-mode fiber for an optical fiber, and it is also desirable that an optical fiber has a winding part 101c that is as long as at least one lap or longer. This enables a more homogenous light illumination; therefore, an analysis apparatus capable of measuring the component concentration inside the biological body with higher accuracy can be realized.

It is also desirable that the specimen contact part 801 is made of a material made up of a metal such as iron, aluminum, and copper, or is made of a material such as diamond and graphite that has high heat conductivity. With this, it is possible to lower the temperature of the biological body 104 with higher speed. This enables the analysis apparatus to improve the measurement speed; therefore, it is preferable to use the above-described materials for the specimen contact part 103.

It is also desirable that the specimen contact part 801 has a concavo-convex shape to fit the biological body, and this enlarges the area contacting the biological body 104. This further enables measurement with higher speed.

As has been described in Embodiment 1 with reference to FIG. 2, the analysis apparatus may be configured to illuminate, using a specimen contact part having high light transmissibility, the biological body 104 with the selective heating light 105 through the specimen contact part.

With this configuration, the temperature easily increases for its high light intensity and the heat is removed from the surface of the biological body, which is to be illuminated with the selective heating light 105 and is the part where an amount of blood flow easily increases. As a result, this enables a more homogenous reduction of the temperature inside the biological body. It is therefore possible to homogenously reduce the amount of blood flow in the whole area of the specimen from the part that is close to the surface to be illuminated with light to the deep portion of the biological body. That is, a more comprehensive and highly accurate measurement of component concentration can be achieved.

The configuration of the analysis apparatus 8 in FIG. 8 is desirable in that it is possible to choose, for the specimen contact part 801, an inexpensive material such as copper and aluminum that has a high light transmissibility, because high light transmissibility is not required for the specimen contact part 801.

For a specimen contact part which requires high light transmissibility, a material having a high thermal resistance and a high transmissibility to transmit the selective heating light 105, such as quartz and diamond, is desirable as a material of the specimen contact part 201. The diamond, in particular, has high heat conductivity and is desirable as the material of the specimen contact part according to the present embodiment.

As is the same as the configuration in FIG. 8, by having a temperature measurement unit, a highly reproducible measurement can be further achieved. Moreover, in the case of cooling from the surface to be illuminated with the selective heating light, it is more desirable to use a transparent temperature measurement unit or a radiation thermometer. Thus, it is possible to measure the surface temperature of the biological body irrespective of the contact between the biological body and the specimen contact part (contact thermal resistance), and what is more desirable is that a response speed is high.

It is possible to more homogenously illuminate the biological body with the selective heating light 105; therefore, the measurement of component concentration distribution with higher accuracy can be achieved.

As described in Embodiment 1 with reference to FIG. 3, it is more desirable to have the specimen contact part inserted between the ultrasonic probe 102a and the biological body 104 because it is possible to more homogenously heat or cool the inside of the biological body 104, and the analysis apparatus capable of a highly accurate measurement can be realized.

As is the same as the configuration in FIG. 8, it is a desirable configuration to have a temperature measurement unit such as a thermistor because a highly reproducible measurement can be achieved.

As has been described in Embodiment 1 with reference to FIG. 3, it is more desirable to set a sound velocity heat changing member, with which a sound velocity changes by temperature change, to a location where an ultrasonic pulse radiated from the ultrasonic probe 102a passes.

With this configuration, merely by measuring the time at which an ultrasonic pulse passes the sound velocity heat changing member with the use of the ultrasonic measurement apparatus 102, it is possible to obtain the temperature of the specimen contact part.

As an example of the sound velocity heat changing member, it is desirable to use a material with which a sound velocity greatly changes due to temperature change, as has been described in Embodiment 1. For example, materials such as rubber and resin can be used as the material of the sound velocity heat changing member. These materials are desirable because a low-cost and lightweight ultrasonic probe can be realized.

Moreover, it is also desirable to use a material having a glass-transition point that is close to a normal temperature for the material of the sound velocity heat changing member. This is because a sound velocity change due to temperature change is large, and a highly accurate measurement can be achieved.

It should be noted that in the case of having the sound velocity heat changing member between a biological body and an ultrasonic probe, it is desirable to use a material having acoustic impedance which is different from those of the biological body and the ultrasonic probe. It is especially desirable to use a material having acoustic impedance of 1.4×10⁶ kg/m²s or smaller, or 1.6×10⁶ kg/m²s or greater.

This generates a greater reflection of the ultrasonic pulse in the boundary surface between the sound velocity heat changing member and the biological body and in the boundary surface between the sound velocity heat changing member and the ultrasonic probe. Thus it is possible to measure the temperature with high accuracy.

It is also desirable to reduce the reflection to a certain extent or smaller, and it is more desirable that the sound velocity heat changing member has acoustic impedance of (1.0 to 1.4)×10⁶ kg/m²s or (1.6 to 2.25)×10⁶ kg/m²s, which realizes a more sensitive ultrasonic probe.

For example, a mixture of polyethylene, silica, and acryl can be used for the material of the sound velocity heat changing member.

This is desirable because it is possible to realize a more inexpensive temperature measurement means compared to the case of using a thermistor or a radiation thermometer, and it is possible to provide a low-cost analysis apparatus.

As has been described in Embodiment 1 with reference to FIG. 4, it is more desirable to have an optical fiber equipped with a fiber grating within the area which is to be illuminated with the selective heating light. The fiber grating can be used as a temperature measuring means by monitoring the wavelength of the reflecting light.

Moreover, with the use of the fiber grating as a temperature measuring means, it is possible to set the temperature measuring means in the part where light and ultrasonic waves pass, which achieves the adjustment of temperature with high accuracy. Namely, this enables further minimization of the variability among measurements due to variability among the temperatures obtained in the respective measurements.

It is also desirable that the temperature of the specimen contact part should be controlled to be −4 degrees Celsius or higher at the time when the internal structure of the biological body is measured by the ultrasonic measurement apparatus. Thus, it is possible to prevent the skin of the specimen from getting frostbitten.

Moreover, it is more desirable that the temperature of the specimen contact part should be controlled to be 15 degrees Celsius or higher. In this case, it is possible to provide the cells with a necessary amount of oxygen. Therefore, the biological body does not easily feel the fatigue caused by a drop in the body temperature even if the measurement is performed for a long time.

It is also desirable that the temperature of the specimen contact part should be controlled to be 25 degrees of Celsius or lower. In this case, it is possible to cool the biological body without being affected by an individual difference in body temperature.

In the case of illuminating the selective heating light through the specimen contact part, it is desirable that the temperature of the specimen contact part is controlled to indicate a room temperature or higher. This can prevent the generation of dew condensation on the specimen contact part, and can restrain the inhomogeneity in the illumination of the biological body with the selective heating light, which is caused by the dew condensation. That is to say that a highly reproducible light illumination can be achieved and the variability among the accuracies in the respective measurements can be minimized. In addition, it is also desirable in the case above that the temperature of the specimen contact part indicates 30 degrees Celsius or lower. This can restrain the inhomogeneity in the illumination of the biological body with the selective heating light, which is caused by the sweating on the skin surface. It is thus possible to achieve a highly reproducible light illumination and minimize the variability among the accuracies in the respective measurements.

In order to prevent the influence caused by individual difference in sweating temperature depending on race, sex, humidity, and others, it is desirable to adjust the temperature of the specimen contact part after the measurement of a sweating temperature of a biological body being a specimen, in order that the temperature of the specimen contact part does not go beyond the sweating temperature that has been measured.

With the use of an ultrasonic probe for which a transparent piezoelectric material is used, it is possible to illuminate a biological body with both light and ultrasonic waves from the same place. By using an ultrasonic probe for which crystal that is a transparent piezoelectric material and bulk-type transparent piezoelectric materials such as lithium niobate and lithium tantalate are used, both the transmission of the ultrasonic waves by the ultrasonic probe and the light illumination on the contact surface between the ultrasonic probe and the biological body can be achieved with low cost. This is desirable because the light intensity of the biological body near the ultrasonic probe becomes more homogenous and stronger, and a more highly accurate and sensitive measurement can be achieved.

It is desirable to use a transparent piezoelectric material utilizing single crystal thin film technology, such as ZnO and AIN, because a more compact analysis apparatus can be realized.

It is further desirable to use an ultrasonic probe which applies a voltage to a piezoelectric material using a transparent electrode such as ITO that has excellent light transmissibility characteristics. This achieves a more sensitive and a highly accurate measurement of component concentration.

Moreover, it is further desirable to use a transparent electrode made of zinc oxide series or magnesium. This achieves, with low cost, a sensitive and highly accurate measurement of the component concentration.

As described in Embodiment 1 with reference to FIG. 5, it is desirable to illuminate the biological body that is set in the water in a tank with the selective heating light and to send/receive the ultrasonic pulses using the ultrasonic probe. Thus, it is possible to keep the temperature of the whole body more homogenous, and to measure the sound velocity temperature change coefficients with higher accuracy. This enables a much highly accurate measurement of component concentration.

It is desirable to set the temperature of the water to 15 degrees of Celsius or higher. Thus, the amount of blood flow which enables the provision of oxygen necessary for the cells inside the biological body is maintained. Therefore, the biological body does not easily feel the fatigue caused by a drop in the body temperature even the measurement is performed for a long time.

It is also desirable to set the temperature of the water to 25 degrees of Celsius or lower. Thus, it is possible to cool the biological body without being affected by individual difference in body temperature.

Although the water is used here, it shall not necessarily be water. It is, however, desirable to use a liquid that is relatively runny. With such liquid having a relatively low viscosity, it is possible to effectively cool the biological body by the heat convected; therefore, a highly accurate measurement of component concentration can be achieved.

Besides water, ethanol may be used, for instance. Ethanol is highly effective in sterilization and there is no need to mix any antiseptic agent.

In the case of using water, a low-cost analysis apparatus can be realized. Water is desirable as it has a reflective index and acoustic impedance both of which are nearly the same as those of a biological body, and highly effective illumination of both light and ultrasonic waves can be achieved. It is also possible to perform the measurement without directly pressing the ultrasonic probe 102a against the biological body, and the shape of the biological body will not be deformed by directly pressing the ultrasonic probe against the biological body. Water is desirable because it is possible to draw comparison with higher accuracy even in the comparison with the results of the measurements performed in the past.

It should be noted that in the case of using water, it is desirable to use water mixed with an antiseptic agent, and a highly reproducible measurement of component concentration can be achieved.

It is more desirable to use water mixed with a surface-activating agent. The use of such water inhibits the formation of blisters on the surface of the biological body and achieves a highly accurate measurement of component concentration.

The present embodiment has described an example of an analysis apparatus which measures a fat concentration; however, the present embodiment is not limited to this and can be applied to all of the component concentration measurements in which a phenomenon of optical heating is put into practice. For example, with the use of the light having a wavelength of 650 nm to 800 nm, it is possible to realize an analysis apparatus which measures oxygen saturation of hemoglobin (a ratio between the concentration of oxidized hemoglobin and the concentration of deoxidized hemoglobin). Furthermore, the present embodiment is applicable for use in a judgment between cancer and benign tumor and for a diagnosis on a depth of burn injury.

In either of the cases of measuring a fat concentration and of measuring concentration and oxygen saturation of hemoglobin, it is desirable to obtain optical absorption of light having plural wavelengths using a light source that generates such light having plural wavelengths, which further enables a measurement of component concentration with high accuracy.

Furthermore, the present embodiment may be applied to an analysis apparatus which targets something other than a biological body. For example, other applications such as an examination of foreign substances contained in food and a detection of the concentration of the components included in gas.

The present embodiment has described the analysis apparatus which measures heating generated with light, using ultrasonic waves; however, the analysis apparatus according to the present invention shall not necessarily use the ultrasonic waves. For example, even with the analysis apparatus which measures temperature change due to optical heating, using a radiation thermometer, it is possible to obtain the effect of reducing the measurement errors caused by the fact that the radiation specter measured by a thermocouple varies depending on the material composition. It is desirable to use a radiation thermometer because a non-contact measurement of component concentration can be achieved.

As has already been described in the present embodiment, the analysis apparatus utilizes a sound velocity change due to temperature increase, and measures heating generated with light, with the use of the ultrasonic waves which are an inexpensive means having an excellent property to directly advance inside the biological body. Such analysis apparatus is desirable as it realizes a measurement of component concentration (distribution) with low cost and an excellent position resolution even inside the biological body.

The present embodiment has described the configuration in which a specimen contact part is set between an ultrasonic probe and a specimen (biological body). However, it may be multi-configured in such a manner that the surface of the ultrasonic probe, at which the ultrasonic probe contacts the biological body, may have a function as the specimen contact part to heat (cool) the biological body.

Furthermore, in the present embodiment, the analysis apparatus is configured in such a manner to include a means to control an amount of blood flow as shown in Embodiment 1, which further enables a highly accurate measurement of component concentration.

### Embodiment 4

As is the same as Embodiment 3, the present embodiment shows an example of an analysis apparatus which heats a specimen with light having a specified wavelength and measures, as a sound velocity change, a difference in an amount of temperature increase due to a difference in optical absorption among respective parts of the specimen. The analysis apparatus restrains the reduction of the measurement accuracy, which is caused by the fact that a proportionality coefficient between a temperature change and a sound velocity change varies depending on a material constitution and a composition ratio of the respective parts of the specimen.

As has been described in Embodiment 3, by comparing the results of the ultrasonic sound velocity measurements between the case when homogenous heating is performed and the case when homogenous heating is not performed, and obtaining sound velocity change coefficients, it is possible to measure a temperature increase distribution (component concentration distribution) that reflects more the actual state, based on a sound velocity change between the case when selective heating light is illuminated and the case when such light is not illuminated (homogenous cooling is not allowed in the present embodiment).

It should be noted that a homogenous heating means is different from that described in Embodiment 3.

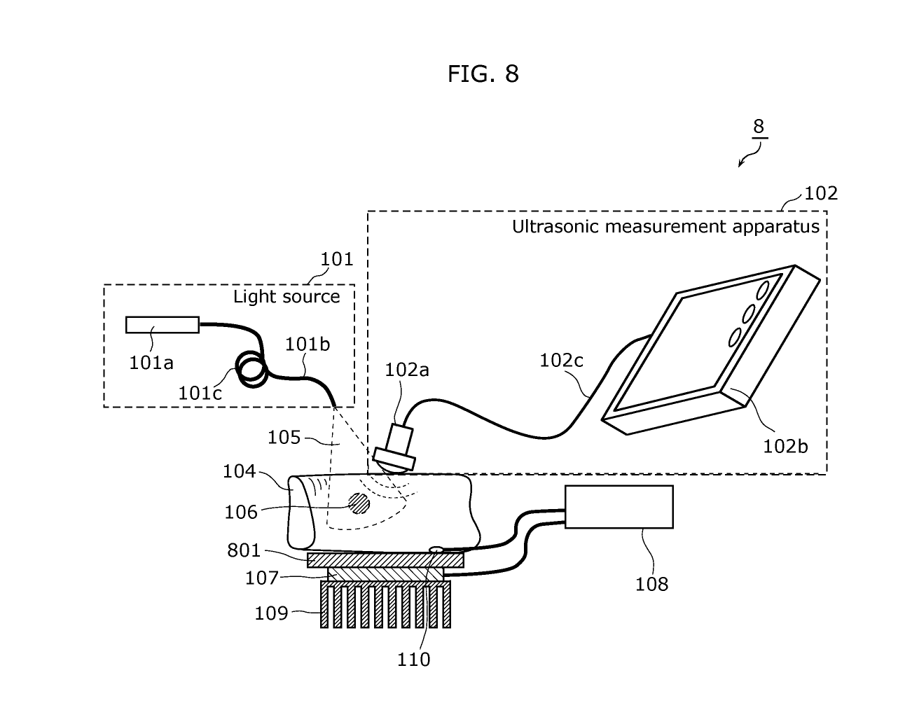

As shown in FIG. 9, according to the analysis apparatus in the present embodiment, homogenous heating is performed by irradiating the microwaves generated in a microwave source 901 on the biological body 104.

The microwaves can be a homogenous heating means as they generate less variability in optical absorption depending on the material constitution in each part inside the biological body 104, compared to far-red light (wavelength is 600 nm to 1500 nm) used as a selective heating light.

In the case where a biological body is a specimen, for instance, it is desirable to irradiate the microwaves of about 2.45 GHz which ensure high water absorption. To be more precise, it is desirable to irradiate the microwaves of 2 to 3 GHz on the biological body because this enables homogenous heating.

Moreover, the irradiation of the microwaves of 3 to 7 GHz or 1 to 2 GHz on the biological body is desirable because it realizes homogenous heating of the biological body to a deeper portion compared with the case of irradiating the microwaves of 2 to 3 GHz.

Thus, the analysis apparatus according to the present embodiment enables a highly accurate measurement of component concentration with the same operation as described in Embodiment 3.

The homogenous heating means using the microwaves as described in the present embodiment enables high accuracy even in the case of using the spectroscopic measurement apparatus which measures component concentration based on elastic wave energy, as shown in FIG. 15.

The analysis apparatus in FIG. 9 can be also applied to the reduction of the variability in proportionality coefficients between elastic waves and optical absorption (reduction in measurement accuracy) which is caused by the fact that a temperature increase ratio and an expansion ratio which are caused by the generation of heat vary depending on a material constitution or a constituent ratio of each part. This can be achieved by pulse driving both a light source and a microwave source and thereby comparing the location where elastic waves are generated by the illumination of differential heating pulsed light and the location where the elastic wave energy is generated by the illumination of pulsed microwaves.

Thus, in the present embodiment, the analysis apparatus is configured to include a means to reduce the blood flow, as described in Embodiment 1, which further enables a highly accurate measurement of component concentration.

Furthermore, with the analysis apparatus configured to include a means to cool a specimen, as described in Embodiment 1, it is possible to irradiate high-power microwaves on the biological body, which further enables a highly accurate measurement of component concentration.

### Embodiment 5

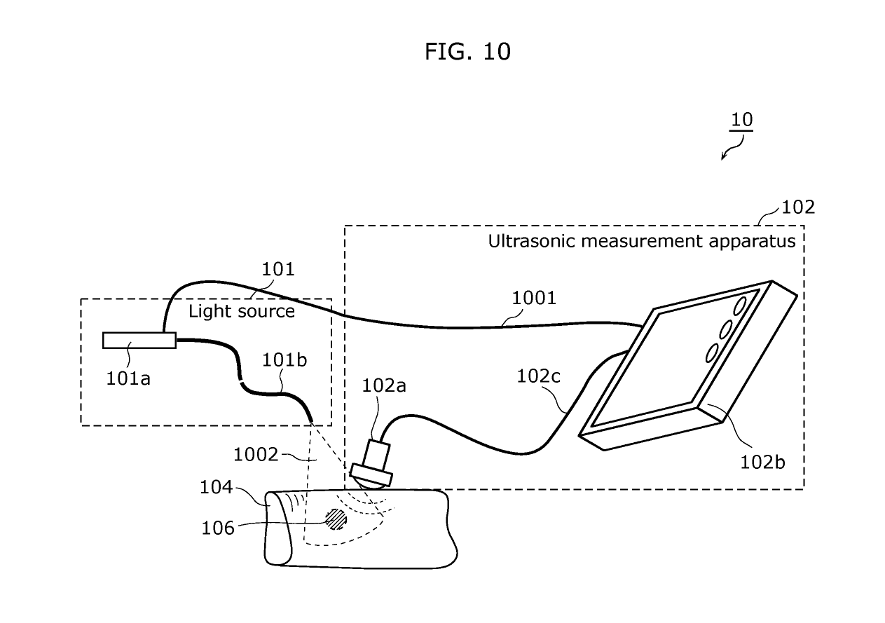

With the spectroscopic measurement apparatus as shown in FIG. 14, a selective optical heating 1102 through the illumination with a selective heating light is started after an ultrasonic sound velocity measurement (first time) 1101 has been performed, as shown in FIG. 11. Then, a temperature 1105 of the plaque 106 inside the biological body 104 is sufficiently increased, and an ultrasonic sound velocity measurement (second time) 1103 is performed at the timing when the alteration of temperature from moment to moment becomes small because heat generation and heat release become balanced.

However, it takes a long time until the heat generation and the heat release become balanced, and the biological body 104 and the ultrasonic probe 102a can be misaligned between the ultrasonic sound velocity measurement (first time) 1101 and the ultrasonic sound velocity measurement (second time) 1103, which has caused degradation in the measurement accuracy.

According to the present embodiment, with the analysis apparatus which heats a specimen with light having a specified wavelength and measures, as a sound velocity change, a difference in temperature increase due to a difference in optical absorption among the respective parts of the specimen, it is possible to shorten the measurement time.

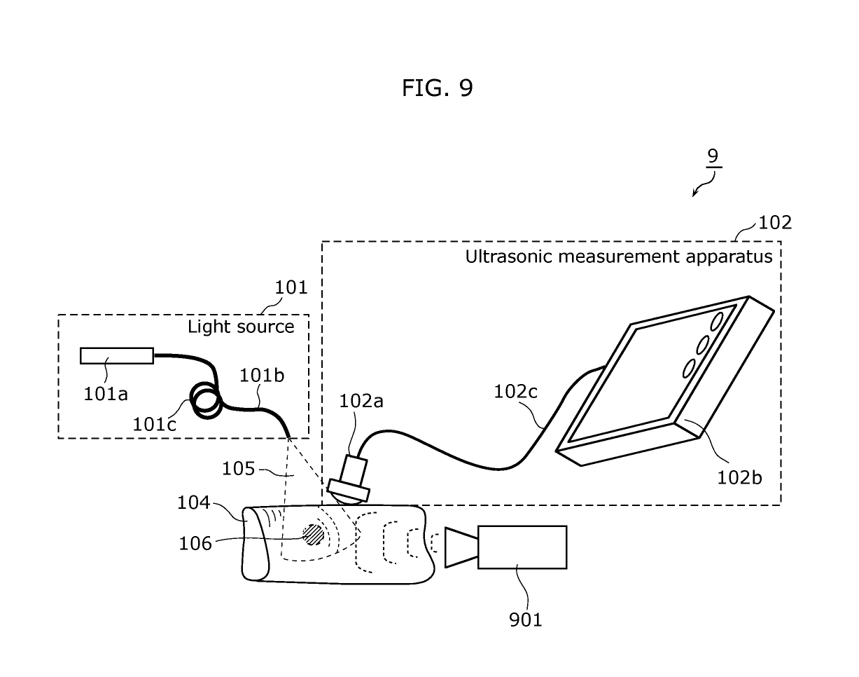

FIG. 10 shows an example of an overall configuration of an analysis apparatus 10 in the present embodiment.

As shown in FIG. 10, the analysis apparatus 10 according to the present embodiment includes the light source 101, the ultrasonic measurement apparatus 102, and a signal transmission line 1001.

The analysis apparatus 10 in FIG. 10 illuminates the biological body 104 with the selective heating light 1202 having a wavelength of 1100 nm or longer and 1300 nm or shorter, and more preferably, a wavelength of about 1200 nm, and measures an amount of fat (fat concentration) of the intravascular plaque 106, as in the conventional case.

By measurement and comparison of the sound velocity in each part inside the biological body 104 between the case when the selective heating light 1002 is illuminated from the light source 101 and the case when such illumination is not performed, it is possible to obtain a sound velocity change (temperature change) inside the biological body 104. Thus, it is possible to obtain a desired component concentration distribution inside the biological body 104.

With the analysis apparatus 10 according to the present embodiment, in particular, the light source 101 and the ultrasonic measurement apparatus 102 are connected via the signal transmission line 1001, which enables precise adjustments of the timing to illuminate the biological body 104 with the selective heating light 1002 and the timing to measure the sound velocity inside the biological body 104 through the transmissions of the ultrasonic pulses to and from the biological body 104.

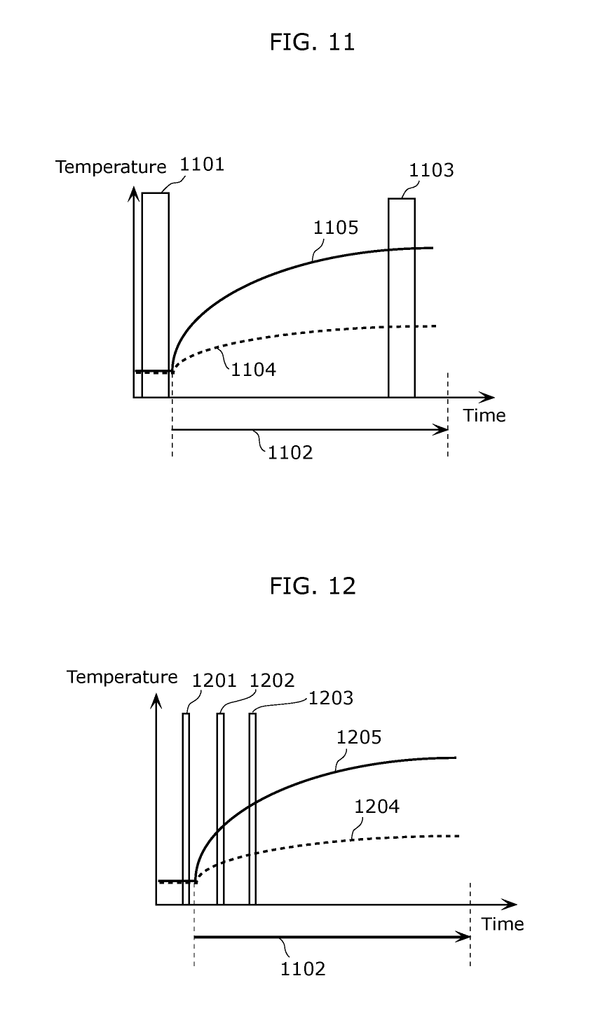

With the analysis apparatus 10 according to the present embodiment, it is desirable to shorten the time between the ultrasonic sound velocity measurement (first time) 1201 (or start of the selective optical heating 1102) and the ultrasonic sound velocity measurement (second time) 1202, as shown in FIG. 12, for instance. This reduces the decrease in the measurement accuracy due to the misalignment of the biological body and the ultrasonic probe, or the like, and enables a highly accurate measurement of component concentration.

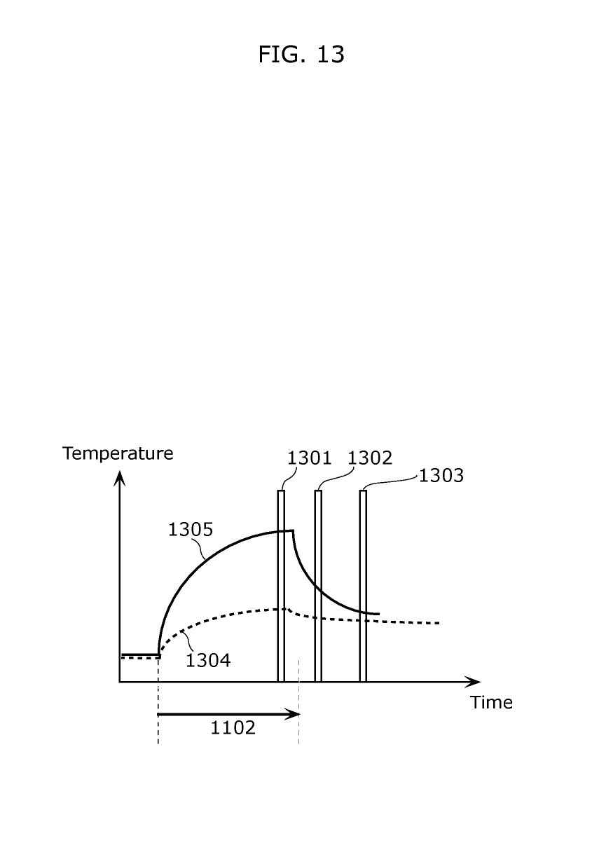

As shown in FIG. 13, it is more desirable to perform the ultrasonic sound velocity measurement (first time) 1301 shortly before the selective optical heating 1102 is ended and perform the ultrasonic sound velocity measurement (second time) 1302 shortly after the selective optical heating 1102 is ended. The temperature 1304 of the part (periphery) other than the plaque 106 increases as well shortly after the start of the selective optical heating 1102. However, shortly after the end of the selective optical heating 1102, the reduction of the temperature 1304 of the periphery is smaller than the reduction of the temperature 1305 of the plaque 106. Therefore, with the ultrasonic sound velocity measurement performed at the timing as shown in FIG. 13, a highly-contrasted measurement of component concentration can be achieved.

Moreover, as shown in FIGS. 12 and 13, in the case of performing the ultrasonic sound velocity measurement at the timing when the time change of the temperature 1205 (or 1305) of the plaque 106 is large, it is desirable to use, for the ultrasonic probe 102a, a ultrasonic probe that is either one of a convex type, an electronic sector-scanning type, and an electronic linear scanning type, or an ultrasonic probe in which transducers are two-dimensionally arranged. In such case, a high-speed ultrasonic sound velocity measurement enables a measurement of component concentration with higher accuracy.

In the case of using an ultrasonic probe that is a convex type or an electronic sector-scanning type, it is desirable to irradiate the ultrasonic pulses while discontinuously changing a direction of ultrasonic beams. This enables a high-speed ultrasonic sound velocity measurement, with which a highly accurate measurement of component concentration can be achieved.

In the case of performing ultrasonic sound velocity measurement shortly after the start or the end of the selective optical heating 1102, as shown in FIGS. 12 and 13, a time constant of the change of the temperature 1205 (or 1305) varies depending on the size of the plaque 106 and the amount of blood flow in the periphery of the plaque 106. Therefore, it is desirable to perform the ultrasonic sound velocity measurement (third time) 1203 or 1303 within ten seconds after the ultrasonic sound velocity measurement (second time) 1202 or 1302. This allows an obtainment of a time constant for each plaque, which further enables a highly accurate measurement of component concentration.

In addition, it is desirable to reverse a direction of ultrasonic beam scanning between the ultrasonic sound velocity measurement (second time) and the ultrasonic sound velocity measurement (third time), which further enables a highly accurate measurement of component concentration.

Moreover, it is desirable that the waveforms of the ultrasonic pulses oscillated in the ultrasonic sound velocity measurement (second time) are different from those of the ultrasonic pulses oscillated in the ultrasonic sound velocity measurement (third time). This further enables a high definition measurement of component concentration, covering the range from a shallow part to a deep part.

With the analysis apparatus that targets a human body as a specimen, it is desirable to perform the ultrasonic sound velocity measurement for plural times within twenty seconds, and obtain a sound velocity change distribution by comparing at least two results of the ultrasonic sound velocity measurements. Twenty seconds is a time during which one can stop breathing irrespective of an individual difference, and it is possible to prevent the measurement errors due to breathing.

The present embodiment has shown an example of the analysis apparatus that measures a fat concentration of a plaque; however, the same effects can be gained with the analysis apparatus which has the same configuration as that of the present embodiment and which aims to measure a concentration of other component or to measure a component concentration distribution.

Furthermore, it is desirable to combine the configurations described in Embodiments 1, 3, 4, and 5 because such combination makes a highly-effective configuration.

Thus, the analysis apparatus and the light illumination apparatus according to the present invention have been described; however, the configurations described in the present description are merely exemplary, and as long as they do not depart from the essence of the present invention, various modifications are possible.

It should be noted that, in each of the embodiments, each constituent element may be configured in dedicated hardware or may be realized by executing a software program intended for each constituent element. Each constituent element may be realized by a program execution unit such as a CPU or a processor by reading and executing the software program recorded onto a recording medium such as a hard disk or a semiconductor memory. Here, the software for realizing the analysis apparatus in each of the above-described embodiments is a program as such that is described below.

Namely, the aforementioned program is an analysis method for analyzing a state of a specimen and causes a computer to execute (Claim 22).

Although the analysis apparatus according to one or more aspects of the present invention has been described up to this point based on the embodiments, the present invention is not limited to these embodiments. As long as they do not depart from the essence of the present invention, various modifications obtainable through modifications to the respective embodiments that may be conceived by a person of ordinary skill in the art as well as an embodiment composed by the combination of the constituent elements of different embodiments are intended to be included in the scope of one or more aspects of the present invention.

### INDUSTRIAL APPLICABILITY

The analysis apparatus according to the present invention is applicable to a measurement of a fat concentration in a liver, an intravascular diagnosis of plaque characteristics, a diagnosis of tumor characteristics, or a measurement of a gas component distribution. The present invention can be a useful means to improve accuracies in these measurements.

### REFERENCE SIGNS LIST

1, 2, 3, 4, 5, 8, 9, 10 analysis apparatus
1a, 101 light source
1b, 110 first temperature measurement unit
1c temperature adjustment unit
1d analysis unit
1e storage unit
6, 7 light illumination apparatus
101a laser light source
101b, 1501b optical fiber
101c winding part of an optical fiber
102, 1502 ultrasonic measurement apparatus
102a ultrasonic probe
102b main body of the measurement apparatus
102c cable
103, 201, 301, 801 specimen contact part
104, 603 biological body
105, 602, 1002 selective heating light
106 plaque
107 heat exchange unit
108 driving electric source
109 heat release unit
302 sound velocity temperature changing member
401 fiber grating
501 tank
502 water
601 cancer tissue
901 microwave source
1001 signal transmission line
1101 ultrasonic sound velocity measurement (first time)
1102 selective optical heating
1103 ultrasonic sound velocity measurement (second time)
1104, 1204, 1304 periphery temperature
1105, 1205, 1305 plaque temperature
1201 ultrasonic sound velocity measurement (first time)
1202 ultrasonic sound velocity measurement (second time)
1203 ultrasonic sound velocity measurement (third time)
1301 ultrasonic sound velocity measurement (first time)
1302 ultrasonic sound velocity measurement (second time)
1303 ultrasonic sound velocity measurement (third time)
1501 pulsed light source
   1501a pulsed laser light source
1502 differential heating pulsed light

1. An analysis apparatus which analyzes a state of a specimen, the apparatus comprising:
a temperature adjustment unit configured to lower a temperature of the specimen by cooling the specimen;
a light source which heats at least a part of the specimen cooled by the temperature adjustment unit, by illuminating the specimen with light;
a first temperature measurement unit configured to measure a change in the temperature of the specimen caused by the heating by the light source; and
an analysis unit configured to analyze the state of the specimen based on the temperature change of the specimen.

2. The analysis apparatus according to claim 1,
wherein the first temperature measurement unit includes:
an ultrasonic probe which transmits an ultrasonic pulse to the specimen and receives a reflected wave that is the ultrasonic pulse reflected from the specimen; and
an ultrasonic wave analysis unit configured to measure the temperature of the specimen based on a signal of the reflected wave received by the ultrasonic probe,
the analysis apparatus further comprises
a storage unit configured to store, into a memory unit, the signal of the reflected wave received by the ultrasonic probe, and
the ultrasonic wave analysis unit is configured to measure the temperature of the specimen based on the signal of the reflected wave stored in the memory unit.

3. The analysis apparatus according to claim 1,
wherein the first temperature measurement unit includes an ultrasonic probe which receives an ultrasonic pulse generated by the specimen, when the light source heats the specimen, and
the analysis unit is configured to analyze the state of the specimen based on the temperature change of the specimen and an intensity of the ultrasonic pulse received by the ultrasonic probe.

4. The analysis apparatus according to claim 1,
wherein the first temperature measurement unit is a radiation thermometer.

5. The analysis apparatus according to claim 1,
wherein the temperature adjustment unit includes:
a heat absorption unit which is disposed in a position contacting the specimen and absorbs an amount of heat from the specimen;
a heat exchange unit which is disposed in contact with the heat absorption unit and includes a Peltier element;
a drive electric source which provides the heat exchange unit with a drive power for driving the heat exchange unit; and
a heat release unit which is disposed in contact with the heat exchange unit and includes a fin that releases an amount of heat absorbed by the heat exchange unit from the specimen.

6. The analysis apparatus according to claim 1,
wherein the temperature adjustment unit includes
a heat absorption unit which is disposed in a position contacting the specimen at a surface that is close to the light source, comprises a material that transmits the light, and is configured to absorb an amount of heat from the specimen, and
the light source illuminates the specimen with light via the heat absorption unit.

7. The analysis apparatus according to claim 5,
wherein the specimen is a biological body,
the analysis apparatus comprises
a second temperature measurement unit configured to measure a temperature of the heat absorption unit, and
the temperature adjustment unit is further configured to adjust the drive power based on the temperature of the heat absorption unit measured by the second temperature measurement unit, to bring the temperature of the heat absorption unit within a range of −4 to 30 degrees Celsius.

8. The analysis apparatus according to claim 1,
wherein the light source illuminates the specimen with light that includes plural wavelength components having wavelengths that are different from one another.

9. The analysis apparatus according to claim 3,
wherein the light source illuminates the specimen with a continuous wave laser (CW) light and a short pulsed light at different timings, the short pulsed light having a pulse width of 0.2 nanoseconds or longer and 330 nanoseconds or shorter.

10. The analysis apparatus according to claim 1, further comprising
a multi-mode fiber which guides the light generated by the light source,
wherein the multi-mode fiber includes a winding part that is as long as one lap or longer in a part of the multi-mode fiber.

11. The analysis apparatus according to claim 2, further comprising
a sound velocity heat changing member which is disposed between the ultrasonic probe and the specimen and has an acoustic impedance of (1.0 to 1.4)×10⁶ kg/m²s or (1.6 to 2.25)×10⁶ kg/m²s.

12. The analysis apparatus according to claim 1,
wherein the first temperature measurement unit includes:
an optical fiber that includes a fiber grating; and
a reflection characteristic measurement unit configured to measure the temperature of the specimen by measuring at least one of a peak reflected wavelength of the fiber grating and a reflectance of a predetermined wavelength, as a reflection characteristic.

13. The analysis apparatus according to claim 1, further comprising:
a tank which stores water that includes an antiseptic agent and is used for cooling the specimen,
wherein the temperature adjustment unit is further configured to adjust a temperature of the water in the tank.

14. The analysis apparatus according to claim 2,
wherein the ultrasonic probe includes a piezoelectric body including crystal, lithium niobate, or lithium tantalate.

15. The analysis apparatus according to claim 1,
wherein the specimen is a biological body,
the light source illuminates the specimen with light having a wavelength of 1100 nm or longer and 1300 nm or shorter, and
the analysis unit is configured to measure fat concentration of a predetermined portion inside the biological body as the state of the specimen.

16. The analysis apparatus according to claim 1,
wherein the temperature adjustment unit is further configured to increase the temperature of the specimen by heating the specimen.

17. The analysis apparatus according to claim 16,
wherein the temperature adjustment unit includes a microwave source which heats the specimen by illuminating the specimen with a microwave.

18. The analysis apparatus according to claim 2,
wherein after the light source illuminated the specimen with the light, the ultrasonic probe transmits an ultrasonic pulse to the specimen and receives a first reflected wave that is the reflected wave, and when the light source is illuminating the specimen with the light, the ultrasonic probe transmits an ultrasonic pulse to the specimen and receives a second reflected wave that is the reflected wave, and
the ultrasonic wave analysis unit is configured to measure the temperatures of the specimen based on signals of the first reflected wave and the second reflected wave as the first temperature and the second temperature, respectively.

19. The analysis apparatus according to claim 2,
wherein after the light source illuminated the specimen with the light, the ultrasonic probe receives a first reflected wave and a second reflected wave which are reflected waves from the specimen, and
the ultrasonic wave analysis unit is configured to measure the temperatures of the specimen based on signals of the first reflected wave and the second reflected wave as the first temperature and the second temperature, respectively.

20. The analysis apparatus according to claim 18,
wherein the ultrasonic probe receives the second reflected wave within twenty seconds after the reception of the first reflected wave.

21. The analysis apparatus according to claim 18,
wherein the ultrasonic probe transmits, to the specimen, two ultrasonic pulses having waveforms that are different from each other, and receives the first reflected wave and the second reflected wave as the reflected waves of the two ultrasonic pulses.

22. An analysis method for analyzing a state of a specimen comprising:
lowering a temperature of the specimen by cooling the specimen;
heating at least a part of the specimen cooled in the lowering of the temperature of the specimen, by illuminating the specimen with light;
measuring a change in the temperature of the specimen caused by the heating in the heating of the specimen; and
analyzing the state of the specimen based on the temperature change of the specimen.
